(19) United States
(12) Patent Application Publication
(10) Pub. No.: US 2023/0277192 A1
(43) Pub. Date: Sep. 7, 2023

(54) ACTUATOR HANDLE FOR SURGICAL TOOLS

(71) Applicant: IMPERIAL COLLEGE INNOVATIONS LIMITED, London (GB)

(72) Inventors: Timothy CONSTANDINOU, London (GB); Jinendra EKANAYAKE, London (GB); Matthew L. CAVUTO, London (GB)

(21) Appl. No.: 18/016,774

(22) PCT Filed: Jul. 19, 2021
(86) PCT No.: PCT/GB2021/051845

§ 371 Date: Jan. 18, 2023

(30) Foreign Application Priority Data
Jul. 19, 2020 (GB) 2011106.8

(51) Int. Cl. A61B 17/128; A61B 17/122

(52) U.S. Cl. CPC A61B 17/1285; A61B 17/1227; A61B 2017/00367

(57) ABSTRACT

An actuator handle for a hand-held surgical tool, the actuator handle comprising: a body part adapted to be gripped by a user's hand, the body part having an end effector mounted or mountable thereon; an actuating member alongside the body part and arranged to be operably moveable by a single finger of the said hand, in a substantially linear manner between first and second positions relative to the body part, for actuating the end effector in use; and one or more coupling components coupled to the actuating member, for mechanically transmitting motive force from the actuating member to the end effector upon movement of the actuating member between the first and second positions by the user in use. A surgical clip, for example for use in aneurysm clipping, is also provided, the clip comprising: first and second clipping arms that are mutually openable and closable; biasing means arranged to resiliently bias the first and second clipping arms into a closed configuration; and first and second engagement fixtures respectively coupled to the first and second clipping arms and arranged such that first and second gripping arms of a complementary surgical clip deployment tool can grip the clip and open the clipping arms by applying a lateral force to the first and second engagement fixtures; wherein each of the first and second engagement fixtures comprises at least part of a ball or at least part of a loop or cup, with which each of the arms of said surgical clip deployment tool can engage in a substantially ball-and-socket manner.

### FIELD OF THE INVENTION

This invention relates to hand-held surgical tools, and, more particularly, to actuator handles for such tools. The actuator handles provided by the present disclosure are applicable to surgical tools across a wide range of surgical fields, including but not limited to laparoscopy, endoscopy, neurosurgery (for example, but in no way limited to, aneurysm surgery), ear, nose and throat surgery, and other minimally-invasive surgical procedures.

The invention also relates to surgical clips, for example for use in treating aneurysms (in particular, but not limited to, brain aneurysms) or blood vessel anastomosis. Such surgical clips may be applied using a surgical tool having an actuator handle according to the present disclosure. However, surgical tools having other actuator handles may also be used to apply the present surgical clips.

### BACKGROUND TO THE INVENTION

Surgical procedures are often carried out with the use of specialist hand-held surgical tools. Amongst such tools are those that have some kind of movable end effector at a distal end of the tool, for performing a surgical function such as grasping or cutting, and a proximal handle end by which the tool is held by the user (e.g. a surgeon or other healthcare professional) and by which the end effector is actuated. Examples of such tools include forceps (e.g. for grasping or applying pressure) and scissors (for cutting).

Other examples of such tools include surgical clip applicators, for example for deploying aneurysm clips, or clips for use in treating blood vessel anastomosis. With such applicators the end effector has a pair of opposing mutually-moveable jaws or arms for holding a clip and, through closing or opening the jaws or arms, for causing the clip to open or close. In such a manner the clip can be opened, introduced around an anatomical feature or defect (such as an aneurysm or anastomosis) that is to be clipped, and then closed to deploy the clip.

Existing surgical tools of this nature, such as forceps, scissors or surgical clip applicators, have two substantially-equivalent mutually-opposing handle parts that are brought together or moved apart by the user to operate the end effector. These handle parts are also used to support the tool in the surgeon's hand. The need to hold the tool steady using the handle parts, for the sake of surgical precision, whilst simultaneously moving the handle parts together or apart in order to operate the end effector, leads to an inherent difficulty in operating the tool in a highly controlled, precise manner. As a consequence of not operating the tool in a steady, precise manner, errors can occur during the surgical procedure, which naturally are undesirable and can lead to medical complications or even death of the patient. Such mutually-opposing handle parts can also obstruct the surgeon's view of the surgical site within the patient's body.

To illustrate this by way of example, aneurysm surgery is a fundamental neurosurgical treatment. An aneurysm is a balloon-like bulge caused by localised weakness in an artery or vein (with arterial aneurysms being more common than venous ones). In neurosurgery, brain bleeds as a result of ruptured brain aneurysms represent a serious healthcare challenge. 40% of ruptured brain aneurysms are fatal, with 66% of survivors left with a permanent neurological deficit. Surgical treatment, termed ‘clipping’, involves placing a clip (usually made of metal) at the base or ‘neck’ of the aneurysm. This stops blood flow into the aneurysm, preventing rupture and haemorrhage into the surrounding brain tissue. Treatment for ruptured as well as unruptured aneurysms may alternatively be performed using endovascular technology (i.e. coiling and stenting of aneurysms). However, the latter is associated with a non-trivial recurrence rate (20-40%), and is not feasible for all aneurysms, such as those with complex morphology. Surgical clipping therefore remains the most robust long-term treatment option with a significantly lower risk of recurrence (2-5%), re-rupture and need for re-treatment. Additionally, it is the only alternative in cases where endovascular treatment has failed.

The technical difficulty of performing aneurysm clipping is widely acknowledged. Current surgical tools have remained largely unchanged for the past 40 years as compared to endovascular technology which continues to evolve constantly. Neurovascular surgeons frequently encounter shortcomings in both the ergonomics and reliable operation of existing clip applicators that have substantially-equivalent mutually-opposing handle parts. In particular, excessive movement of the applicator during clipping, clip slippage during removal and re-application, restricted visibility of the surgical site, and limited manoeuvrability, particularly during on-table aneurysm rupture, present serious technical challenges. Risks include inadvertent aneurysm rupture, and excessive surgical manipulation to enable clip application, which can result in disability or death.

Moreover, existing designs of aneurysm clips (and other surgical clips) face problems of non-optimal alignment and the significant concern of clip slippage relative to the applicator tip during intraoperative clip placement and repositioning. This is particularly relevant in cases of increased operative difficulty, as are being increasingly experienced by surgeons due to more complex morphologies of aneurysms being referred for surgery. The latter is compounded by neurovascular surgeons in the modern era having to deal with these cases early on in their surgical careers, as the majority of aneurysms with ‘simple’ morphology are treated by endovascular means.

Therefore, in view of the mortality and morbidity associated with neurovascular surgery, together with an increasing requirement for surgery as a definitive treatment option for morphologically complex and recurrent aneurysms (including post-coiling recurrences), there is a need for a surgical clip applicator that enables more precise (yet nevertheless simple) operation, along with less obstruction of the surgical site, with a view to achieving safer surgery (e.g. neurosurgery). There is also a desire for improved surgical clips (e.g. aneurysm clips) that address at least some of the above problems.

More generally, aside from clip applicators, similar problems are encountered with other surgical tools (such as grasping tools or cutting tools) that have a pair of substantially-equivalent handle parts that are used both to support the device and to operate an end effector. That is to say, it can be inherently difficult to hold the tool steady using the handle parts whilst simultaneously moving the handle parts together or apart in order to operate the end effector, thus resulting in a loss of surgical precision and increasing the risk of an accident occurring.

There is therefore a need for hand-held surgical tools that address at least some of the above problems.

### SUMMARY OF THE INVENTION

According to a first aspect of the present invention there is provided an actuator handle for a hand-held surgical tool as defined in claim 1 of the appended claims. Thus there is provided an actuator handle for a hand-held surgical tool, the actuator handle comprising:
a body part adapted to be gripped by a user's hand, the body part having an end effector mounted or mountable thereon;
an actuating member alongside the body part and arranged to be operably moveable by a single finger of the said hand, in a substantially linear manner between first and second positions relative to the body part, for actuating the end effector in use; and
one or more coupling components coupled to the actuating member, for mechanically transmitting (and optionally amplifying) motive force from the actuating member to the end effector upon movement of the actuating member between the first and second positions by the user in use.

The term “single finger” as used herein should be interpreted broadly, to encompass the possibility of it being the user's thumb.

By virtue of the body part being adapted to be gripped by the user's hand, and the substantially-linear single finger operation of the actuating member, this enables the user (surgeon) to hold the device steady whilst simultaneously actuating the end effector, within minimal disturbance to the end effector during the actuation process.

Preferably the first position is outward relative to the body part and the second position is inward relative to the body part. This leads to a comfortable actuation action for the surgeon, with minimal disturbance to the end effector.

Preferably the actuating member is resiliently biased into the first position. Accordingly, the user is required to apply pressure to the actuating member to overcome this resilient biasing, which gives the actuation process a precise, controlled feel, and may also provide some measure of tactile feedback. Moreover, when the applied pressure is reduced, the resilient biasing will automatically return the actuating member towards its initial state.

In certain embodiments the actuating member comprises a lever arm.

The coupling components may include linkages that, together with the actuating member, form a Scott-Russell linkage mechanism arranged to provide reciprocating motion of a connection point within said linkage mechanism in response to movement of the actuating member between the first and second positions, to which connection point the end effector is coupled in use.

At least a principal part of the Scott-Russell linkage mechanism may form a grippable part of the body part. In this regard, the principal part of the Scott-Russell linkage mechanism may comprise guide regions in which to locate the thumb and middle finger of the user's hand in use, the actuating member being arranged to be operated by the first finger of said hand.

Alternatively, the body part and/or the actuating member may comprise one or more finger rings or finger tangs in which to locate one or more fingers or the thumb of the user's hand in use.

Advantageously the actuating member may be formed in a compliant unitary manner with the principal part. The use of a compliant unitised structure of this kind enables optimal energy-efficient transmission of force and enhanced precision of use. Further advantages that may be realised as a result of such a design include ease of manufacturing, and reduced friction and reduced mechanical wear during use.

Further, a first intermediate linkage may be arranged to couple a pivot end of the actuating member to the principal part. Advantageously, the first intermediate linkage may be formed in a compliant unitary manner with both the actuating member and the principal part.

The first intermediate linkage may comprise flexible waisted regions joining the first intermediate linkage to the actuating member, and joining the first intermediate linkage to the principal part.

Alternatively, the first intermediate linkage may be formed of thinner material than both the actuating member and the principal part, to enable preferential flexure of the first intermediate linkage relative to both the actuating member and the principal part.

Further, a second intermediate linkage may be arranged to couple a point partway along the length of the actuating member to the principal part.

The second intermediate linkage may be formed in a compliant unitary manner with either the principal part or the actuating member.

Advantageously, substantially the entire actuation mechanism, including the actuating member and the rest of the Scott-Russell linkage mechanism, may be formed as a single unitary part. Moreover, the actuator handle may have a skeletal structure with no outer casing to the Scott-Russell linkage mechanism. This advantageously enables a reduction in weight to be achieved, along with increased compactness and improved sterilisation ability.

In certain embodiments the coupling components include a cable that is arranged to move axially, in a reciprocating manner with operation of the actuating member between the first and second positions, for actuating the end effector. If a cable is used, the section of cable in the handle need not travel colinearly with the distal shaft portion of the cable, thereby allowing for flexibility in handle/wrist angle.

In other embodiments the coupling components may include a rod that is arranged to move axially, in a reciprocating manner with operation of the actuating member between the first and second positions, for actuating the end effector.

The cable or rod may be coupled to aforementioned connection point.

The abovementioned second intermediate linkage may include an aperture through which the cable or rod from said connection point passes.

In other embodiments the actuating member may comprise a push-button.

For example, the push-button may be coupled to the rack of a rack-and-pinion mechanism, and the pinion of the rack-and-pinion mechanism may be coupled to the cable.

Alternatively the actuating member may comprise a rotary lever arm, which may be coupled to the cable.

In embodiments for which the coupling components include a cable, the cable may pass around a pulley mechanism to provide mechanical advantage between the actuating member and the end effector. To increase the mechanical advantage the pulley mechanism may comprise one or more nested pulleys.

In certain embodiments, rather than having a skeletal structure, the body part may have an outer casing (either a partial casing or a full casing) to prevent or reduce contact or entanglement with moving parts therein.

Any of the actuator handles herein may further comprise one or more of:
an attachment mechanism for detachably attaching the end effector to the body part;
a rotation mechanism for rotating the end effector relative to the body part;
a locking mechanism for reversibly locking the configuration of the end effector.

According to a second aspect of the invention there is provided a hand-held surgical tool comprising an actuator handle according to the first aspect of the invention, and an end effector attached to, or attachable to, the body part of the actuator handle

According to a third aspect of the invention there is provided a surgical clip, for example for use in aneurysm clipping, the clip comprising:
first and second clipping arms that are mutually openable and closable;
biasing means arranged to resiliently bias the first and second clipping arms into a closed configuration; and
first and second engagement fixtures respectively coupled to the first and second clipping arms and arranged such that first and second gripping arms of a complementary surgical clip deployment tool can grip the clip and open the clipping arms by applying a lateral force to the first and second engagement fixtures;
wherein each of the first and second engagement fixtures comprises at least part of a ball or at least part of a loop or cup, with which each of the arms of said surgical clip deployment tool can engage in a substantially ball-and-socket manner.

In certain embodiments the first and second engagement fixtures are arranged to be urged towards one another to open the clipping arms. However, in alternative embodiments the first and second engagement fixtures may be arranged to be urged apart from one another to open the clipping arms.

The surgical clip may further comprise a hinge portion by means of which the clipping arms are openable and closable.

Advantageously the hinge portion may comprise, or be, the biasing means.

In certain embodiments the hinge portion may comprise:
first and second outer members that are respectively attached to the first and second clipping arms, and to which the first and second engagement fixtures are respectively attached; and
a resilient hinge member between, and coupled to, the first and second outer members, and by means of which the first and second outer members, and thence the first and second clipping arms, can move upon application of lateral force to the first and second engagement fixtures.

For example, the resilient hinge member may be substantially V-shaped, wherein the V-shape is closable upon application of sufficient lateral force to the first and second engagement fixtures, resulting in the clip reaching a state of maximal opening.

In certain embodiments each of the first and second engagement fixtures has a base portion that extends around at least part of the first outer member and the second outer member respectively.

Each of the first and second engagement fixtures may incorporate a reinforcing member.

In certain embodiments, instead of the resilient hinge member being V-shaped, the resilient hinge member may be arc shaped.

The first and second outer members may be arc shaped. In certain embodiments the first and second outer members are relatively inflexible in comparison to the resilient hinge member. However, in other embodiments the first and second outer members may be relatively flexible in comparison to the resilient hinge member.

In certain embodiments the hinge portion and the first and second clipping arms are coplanar, and/or are of unitary form (i.e. having a compliant structure).

Such clips of unitary compliant form may advantageously be formed as (or from) a single piece of material. However, this need not necessarily be the case, as they may alternatively be made from separate materials or separate components that are joined (e.g. fused) together during manufacture. For example, the hinge portion (and the resilient hinge member in particular) may be made of a different material from the clipping arms. Alternatively, the clip could be made in two separate halves that are joined (e.g. fused) together.

In other embodiments the first clipping arm and the first engagement fixture may be part of a first principal component; the second clipping arm and the second engagement fixture may be part of a second principal component; the first and second principal components may be moveable relative to one another; and the biasing means may further comprise a spring to which the first and second principal components are both attached.

In yet other embodiments the hinge portion may be formed as a spring coil to which the first and second engagement fixtures and the first and second clipping arms are attached. In one example the first and second engagement fixtures may be located between the hinge portion and the respective first and second clipping arms.

More generally, each of the first and second engagement fixtures may comprise at least part of a ball, the shape of which is at least partially spherical, or the shape of which is at least partially a geometric solid (other than a sphere) having rotational symmetry.

Alternatively, each of the first and second engagement fixtures may comprise at least part of a loop, the shape of which is at least part of a circle, or the shape of which is at least part of a geometric shape having rotational symmetry, such as a triangle, square, hexagon or octagon.

In certain embodiments the surgical clip may be at least partially made of a transparent (e.g. polymer) material, for example along at least part of the first and second clipping arms. This provides the advantage of enabling the surgeon to verify that the intended anatomical feature is being clipped, and that an unintended anatomical feature is not being clipped (thus, if necessary, allowing the clip to be safely removed and re-deployed before any harm is potentially caused).

In certain embodiments the surgical clip is an aneurysm clip. However, other types of surgical clip are also possible.

According to a fourth aspect of the invention there is provided a surgical clip deployment tool or an end effector for such a tool, comprising first and second gripping arms configured to engage with, and apply a lateral force to, the first and second engagement fixtures of a surgical clip according to the third aspect of the invention.

Optionally the surgical clip deployment tool may have an actuator handle according to the first aspect of the invention.

According to a fifth aspect of the invention there is provided a surgical kit comprising:
a hand-held surgical tool according to the second aspect of the invention (comprising an actuator handle according to the first aspect of the invention, and an end effector); and
one or more surgical clips according to the third aspect of the invention;
wherein the surgical tool or the end effector is in accordance with the fourth aspect of the invention.

### BRIEF DESCRIPTION OF THE DRAWINGS

Embodiments of the invention will now be described, by way of example only, and with reference to the drawings in which:

FIG. 1 illustrates a first embodiment of an actuator handle for a hand-held surgical tool, the handle having a body part adapted to be gripped by a user's hand, a push-button actuating member movable by a single finger of the same hand, for actuating an end effector (not shown), and an optional rotation mechanism for rotating the end effector;

FIG. 2 is a cross-sectional view of the handle of FIG. 1, showing the push-button actuating member connected to a rack-and-pinion mechanism and thence to a cable and pulley arrangement, together forming a force transmission (and magnification) mechanism for operating the end effector;

FIG. 3 is a cross-sectional view of the rotation mechanism of the handle of FIG. 1;

FIG. 4a is a cross-sectional view of a distal end of an end effector mounting shaft, extending from the handle of FIG. 1 and having a rotatable member to which an end effector (not shown) is coupled, and also showing a first arrangement for spring-biasing the end effector;

FIG. 4b is a cross-sectional view of an alternative arrangement for spring-biasing the end effector;

FIG. 5 is a cross-sectional view of a variant of the embodiment of FIG. 1, showing the push-button actuating member connected to an alternative force transmission mechanism;

FIG. 6 illustrates the actuator handle of FIG. 1 in use, with the user's hand gripping and operating the handle using a precision “pencil” grip between their thumb and middle finger, and with their index finger on the actuating member;

FIG. 7 is a cross-sectional view of a second embodiment of an actuator handle for a hand-held surgical tool, the handle having a rotary lever arm actuating member to which a cable and pulley arrangement is connected to form a force transmission (and magnification) mechanism;

FIG. 8 is a cross-sectional view of a third embodiment of an actuator handle for a hand-held surgical tool, the handle having a lever arm actuating member that forms part of a Scott-Russell linkage mechanism to which a cable and pulley arrangement is connected to form a force transmission (and magnification) mechanism;

FIG. 9 is a cross-sectional view of a variant of the actuator handle of FIG. 8, with the lever arm forming part of a modified Scott-Russell linkage mechanism to which a cable is connected to form a force transmission (and magnification) mechanism;

FIG. 10 illustrates a fourth embodiment of an actuator handle for a hand-held surgical tool, the handle having a lever arm actuating member that forms part of a compliant Scott-Russell linkage mechanism to which a cable or rod is connected to form a force transmission (and magnification) mechanism, wherein the compliant Scott-Russell mechanism also forms the body part of the handle;

FIG. 11 illustrates a ball and socket coupling between the cable or rod and the compliant Scott-Russell mechanism of FIG. 10;

FIG. 12a illustrates the actuator handle of FIG. 10 with the lever arm actuating member in a first position, outward relative to the body part;

FIG. 12b illustrates the actuator handle of FIG. 10 with the lever arm actuating member in a second position, inward relative to the body part;

FIG. 13 illustrates the compliant Scott-Russell mechanism of FIG. 10 prior to full assembly;

FIG. 14 illustrates a variant of the compliant Scott-Russell mechanism of FIGS. and 13;

FIG. 15a illustrates the actuator handle of FIG. 10 in use, with the actuating member in the first position (as in FIG. 12a), and the user's hand gripping and operating the handle using a precision “pencil” grip between their thumb and middle finger, with their index finger on the actuating member;

FIG. 15b is a further illustration of the actuator handle of FIG. 10 in use, with the actuating member now in the second position (as in FIG. 12b);

FIG. 15c is a further illustration of the actuator handle of FIG. 10 in use, showing an end-on view of the precision “pencil” grip used in FIG. 15b;

FIG. 16 illustrates a variant of the actuator handle of FIG. 10, prior to full assembly (corresponding to FIG. 13), with the body part and lever arm actuating member having finger rings (or finger tangs) through which to locate one or more fingers or the thumb of the user's hand;

FIG. 17 illustrates the actuator handle of FIG. 16 in use, with the user's hand gripping and operating the handle using a “rope” grip, with their thumb on the actuating member;

FIG. 18a illustrates a surgical clip (e.g. an aneurysm clip) in a closed configuration, the clip having first and second clipping arms and a resilient hinge portion that are coplanar and of unitary form, and first and second ball-like engagement fixtures by which the clip may be gripped and opened using a complementary clip deployment tool;

FIG. 18b shows the clip of FIG. 18a in an open configuration;

FIGS. 18c and 18d are plan views corresponding to the perspective views of FIGS. 18a and 18b;

FIGS. 19a-19d illustrate, in closed and open configurations respectively, a variant of the clip of FIGS. 18a-18d, the variant having first and second loop-like engagement fixtures by which the clip may be gripped and opened using a complementary clip deployment tool;

FIGS. 19c and 19d are plan views corresponding to the perspective views of FIGS. 19a and 19b;

FIG. 20 illustrates the clip of FIGS. 18a-18d being deployed by a complementary clip deployment tool (in this example, an end effector as may be actuated by the actuator handle of any of FIGS. 1-17);

FIG. 21 illustrates the clip of FIGS. 19a-19d being deployed by a clip deployment tool (again, in this example, an end effector as may be actuated by the actuator handle of any of FIGS. 1-17);

FIG. 22 illustrates superimposed examples of angular positions in which the clip of FIGS. 18a-18d may be gripped relative to the clip deployment tool;

FIG. 23 illustrates superimposed examples of angular positions in which the clip of FIGS. 19a-19d may be gripped relative to the clip deployment tool;

FIGS. 24a and 24b illustrate, in perspective and plan views respectively, an alternative configuration of a surgical clip comprising first and second principal components and a biasing spring to which the first and second principal components are both attached, the clip having first and second ball-like engagement fixtures by which it may be gripped and opened using a complementary clip deployment tool;

FIGS. 25a and 25b illustrate, in perspective and plan views respectively, a variant of the clip of FIGS. 24a and 24b, the variant having first and second loop-like engagement fixtures by which the clip may be gripped and opened using a complementary clip deployment tool;

FIGS. 26a and 26b illustrate, in perspective and plan views respectively, an alternative configuration of a clip, wherein the hinge portion is formed as a spring coil to which the first and second engagement fixtures and the first and second clipping arms are attached;

FIGS. 27a and 27b illustrate, in perspective and plan views respectively, a variant of the clip of FIGS. 26a and 26b, the variant having the first and second engagement fixtures located between the spring coil and the respective first and second clipping arms;

FIGS. 28a, 28b and 28c illustrate (in plan view in a closed configuration, in a perspective view in the closed configuration, and in plan view in an open configuration, respectively) a variant of the clip of FIGS. 18a-18d;

FIGS. 29a, 29b and 29c illustrate a variant of the clip of FIGS. 28a-28c, in corresponding views, showing that subtle thinning of certain parts of the hinge portion enables wider opening of the clip; and

FIGS. 30a and 30b illustrate (in plan and perspective views, respectively) a further variant of the clip of FIGS. 18a-18d, in which the hinge portion incorporates an arced (rather than V-shaped) resilient hinge member.

In the figures, like elements are indicated by like reference numerals throughout.

### DETAILED DESCRIPTION OF PREFERRED EMBODIMENTS

The present embodiments represent the best ways known to the Applicant of putting the invention into practice. However, they are not the only ways in which this can be achieved.

The present disclosure provides actuator handles for hand-held surgical tools, and surgical clips. The actuator handles may be used in respect of a range of surgical tools that have some kind of movable end effector at the distal end of the tool, for performing a surgical function such as grasping, cutting, or deploying a surgical clip. Thus, the present actuator handles may advantageously be used in respect of the deployment of the present surgical clips, although the use of the present actuator handles is in no way limited to such a purpose.

Likewise, the present surgical clips may advantageously be deployed using surgical tools having the present actuator handles, although this is by no means necessary and other tools may be used to deploy the present clips.

Embodiments of the present actuator handles and surgical clips will now be described.

Actuator Handles

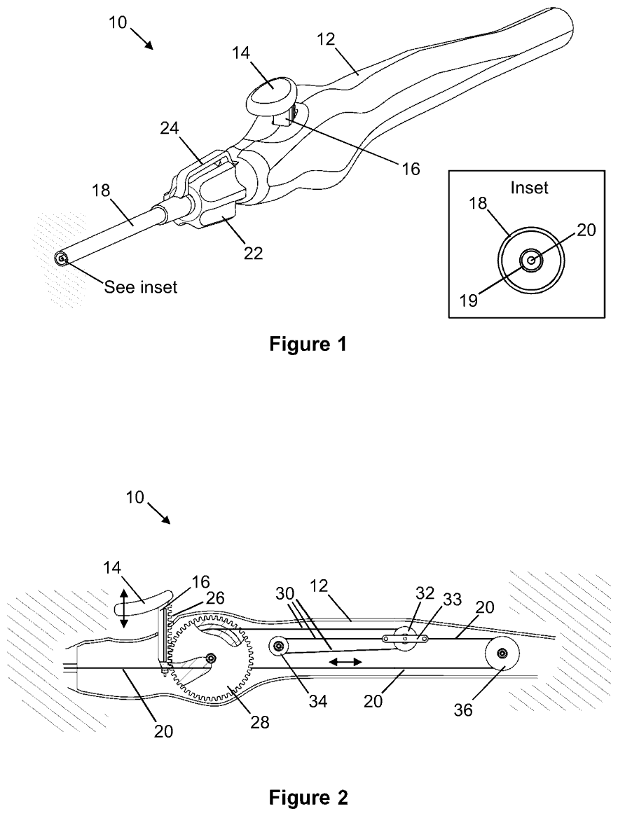

By way of an initial overview, and with reference initially to the embodiment of FIG. 1, the present work provides an actuator handle 10 for a hand-held surgical tool. The actuator handle comprises a body part 12 adapted to be gripped by a surgeon's hand in use. The body part 12 has an end effector (not shown in FIG. 1) mounted or mountable thereon, at the distal end of a hollow shaft 18 that is attached to the body part 12. That is to say, the end effector (and the shaft 18 connecting the end effector to the body part 12) may be formed as an integral part of the handle 10; or alternatively may be a separate component that is attachable to, and detachable from, the handle 10. Indeed, a range of different end effectors to provide different functions (e.g. cutting, gripping, etc.) or of different shapes or sizes may be provided for interchangeable attachment to the handle 10 in a modular fashion according to the surgeon's requirements or preferences, thereby yielding a range of different tools which all employ the same actuator handle 10. Such tools may be used across a wide range of surgical fields, including endoscopic, laparoscopic and minimally invasive surgical procedures.

In some embodiments the shaft 18 may be rigid and straight. In other embodiments the shaft 18 may rigid and curved. In yet other embodiments the shaft 18 may be flexible, permitting the surgeon to bend it during use. For example, the shaft 18 may be made of a shape memory material. In a further alternative the shaft may be rigid and straight, but offset to one side of the central plane of the handle.

Push Button Actuating Member

The actuator handle 10 of FIG. 1 further comprises an actuating member 14 alongside the body part 12 and arranged to be operably moveable by a single finger of the user's hand (the same hand as supports the body part 12), in a substantially linear manner between first and second positions relative to the body part 12, for actuating the end effector. As mentioned above, the term “single finger” should be interpreted broadly, to encompass the possibility of it being the user's thumb. In the illustrated example the actuating member 14 is in the form of a push-button, although other configurations of the actuating member 14 are possible and a number of alternative examples are described herein. In the first position the actuating member 14 is outward relative to the body part 12, whereas in the second position the actuating member 14 is inward relative to the body part 12.

Movement of the actuating member 14 from the first position fully to the second position, or vice versa, is not necessarily a requirement for actuating the end effector, and partial movement of the actuating member 14 between the first and second positions may be sufficient in some instances and for some purposes.

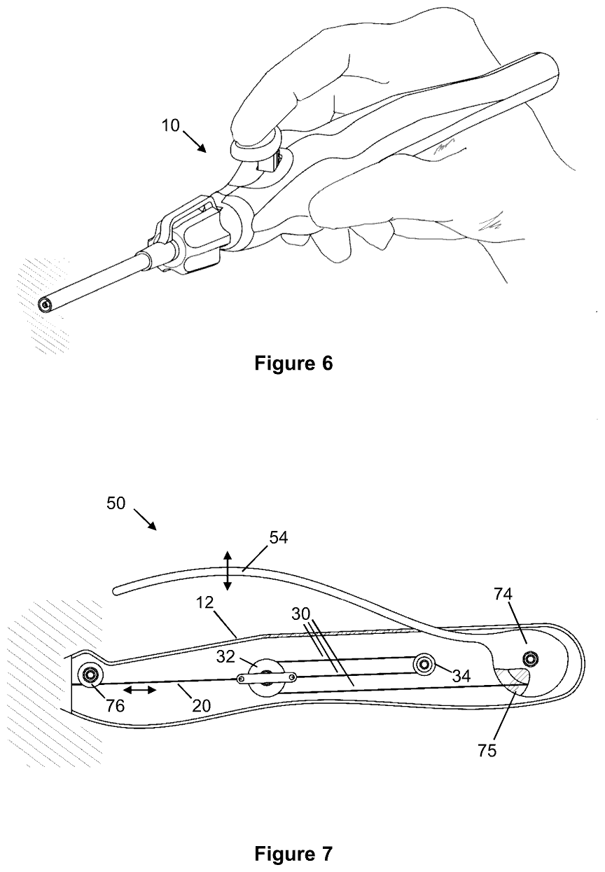

Accordingly, in use (as shown in FIG. 6), the surgeon's hand can hold the body part 12 steady, with the movement of the single finger that operates the actuating member 14 being isolated from the rest of the handle 10 and the rest of the tool in general. As a consequence, the overall tool can be held very steady during surgery, with minimal or no unwanted movement during actuation of the end effector. In other words, by gripping the body part 12, the surgeon is able to hold the entirety of the tool still (with the exception of the actuating member 14, that is moved by the single finger), in a fixed position relative to the surgeon's hand. The surgeon's hand also remains still during the actuation process, with the exception of the single finger that moves to operate the actuating member 14.

The overall stability of the tool is further enhanced by the ability to hold the handle in a precision “pencil” grip, as shown in FIG. 6, whereby the handle is gripped between the surgeon's thumb and middle finger, and their index finger is placed on the actuating member. Such a grip is biomechanically advantageous for small and precise hand movements.

The actuator handle 10 further comprises one or more coupling components coupled to the actuating member 14, for mechanically transmitting motive force from the actuating member 14 to the end effector upon movement of the actuating member 14 between the first and second positions by the user. One exemplary arrangement of such coupling components is illustrated in FIG. 2, whereas alternative arrangements of coupling components are shown in a number of the subsequent drawings.

With the embodiment of FIG. 1, the body part 12 comprises an external case which encloses the moving components of the internal mechanism. Such a case provides the following advantages which have particular relevance to (but are not limited to) minimally invasive surgery:
it prevents interaction and entanglement of the internal parts with external material;
the internal moving parts can be ‘sealed’ from the outside, preventing contamination or clogging; and
a narrowed, low profile cylindrical structure for the external case, as illustrated at FIG. 1, facilitates the introduction of the tool into constrained spaces.

When actuated, movement of the tool is isolated to the actuating member 14 only, with the main body 12 of the tool remaining in a fixed position. The latter is therefore tuned for minimal movement in a constrained anatomical environment.

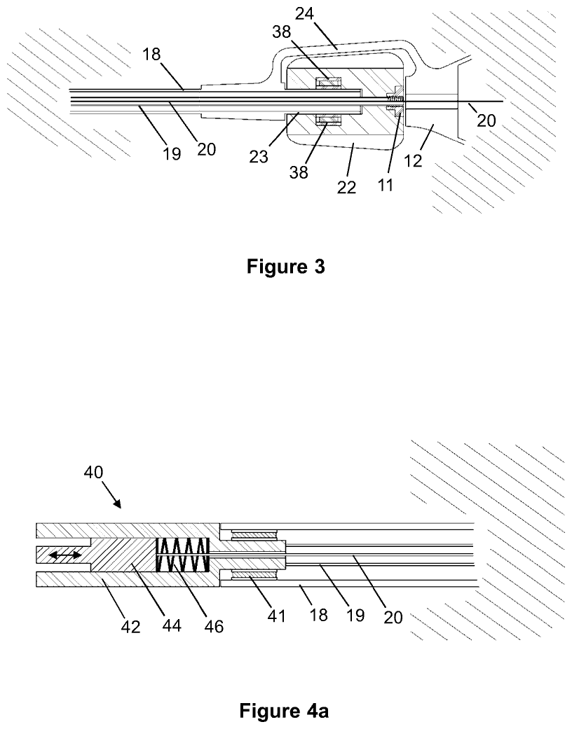

The embodiment of FIG. 1 also includes an optional rotation mechanism (of which rotary adjustment knob 22 is a part) for rotating the end effector relative to the body part 12. This rotation mechanism will be described in greater below, with reference to FIG. 3.

FIG. 2 is a cross-sectional view of the handle 10 of FIG. 1, showing one example of an arrangement of internal coupling components by which motive force can be mechanically transmitted from the actuating member to the end effector in use. As illustrated, the push-button actuating member 14 is provided with a shaft 16 that is slidably mounted within a guide aperture in the casing of the body part 12. The shaft 16 is provided with a toothed rack member 26 that engages with a pinion 28 to form a rack-and-pinion mechanism. A cable 30 is windingly attached to the pinion 28 and passes around a nested pulley mechanism to transmit motive force to a main drive cable 20. In this example the pulley mechanism involves the cable 30 passing from the pinion 28 around a moveable pulley 32 (which is mounted within a moveable block 33), around a first fixed pulley 34, and is then connected to one side of the moveable block 33. The main drive cable 20 is connected to the other side of the moveable block 33 and then passes around a second fixed pulley 36 before passing along the shaft 18 (see FIG. 1 and the inset thereof) towards the end effector. Due to the presence of the moveable pulley 32, the pulley mechanism provides a mechanical advantage (in this case, of a factor of three) in respect of the force transmitted to the end effector. It will of course be appreciated that other pulley arrangements may be employed for a similar purpose.

As the surgeon presses the actuating member 14, moving it from the first position towards the second position, the pinion 28 rotates and winds-in the cable 30, which in turn causes the main drive cable 20 to be pulled in the direction away from the end effector.

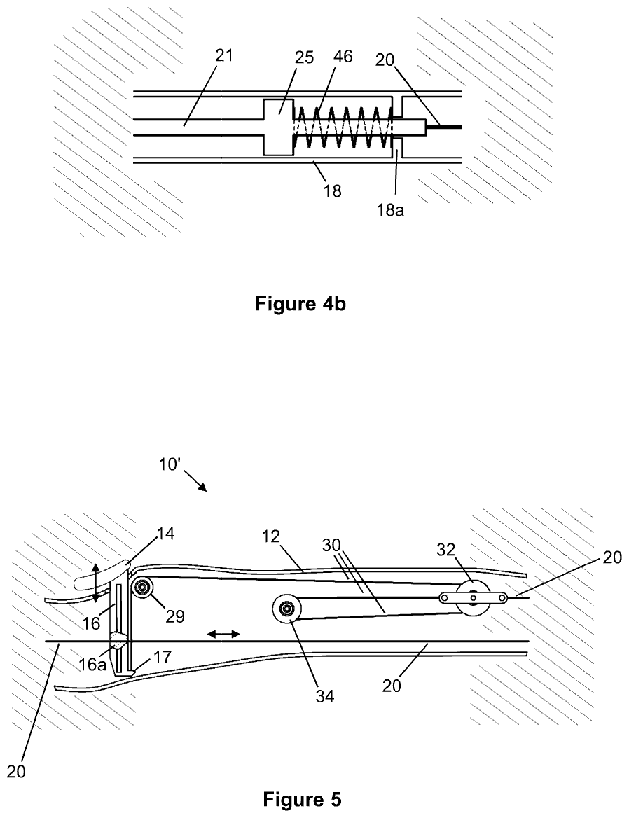

In the mechanism of FIG. 1, and similarly with the other actuation mechanisms described below, the actuating member 14 is preferably resiliently biased into the first position. This may be achieved for example by spring-biasing the end effector mechanism (see e.g. the compression spring 46 in FIGS. 4a and 4b) to pull the drive cable in the direction towards the end effector. The actuating member 14 may also be spring biased.

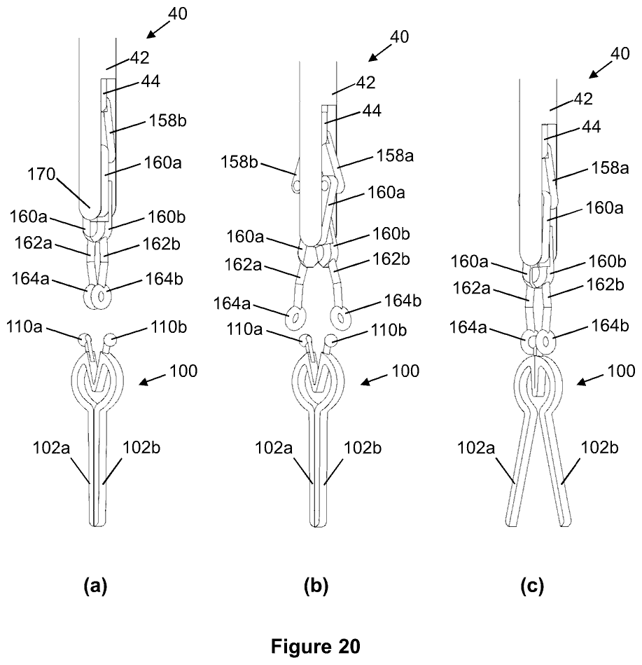

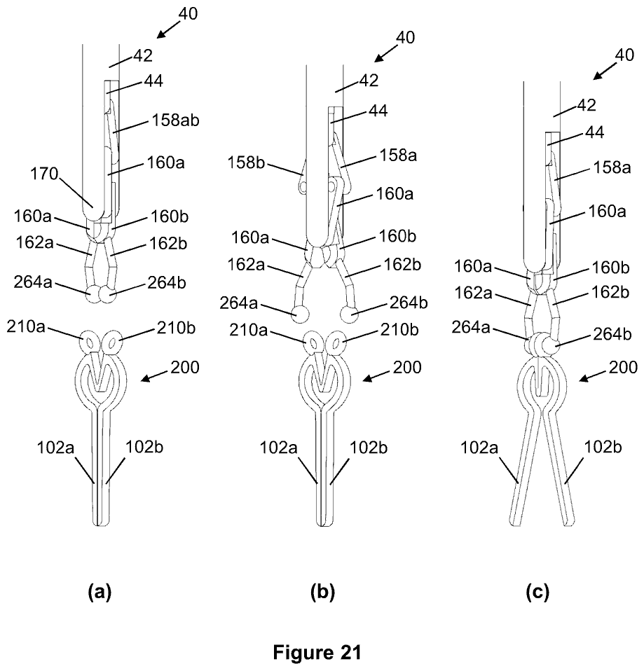

FIG. 3 is a cross-sectional view of the rotation mechanism of the handle 10 of FIG. 1, the rotation mechanism being operable to rotate the end effector relative to the body part 12, by the user turning the rotary adjustment knob 22. Reference is also made to FIG. 4a, which is a cross-sectional view of the distal end 40 of the shaft 18, having a rotatable member 42 which ultimately supports the end effector (not shown in FIG. 4a; examples are shown in FIGS. 20 and 21). The rotatable member 42 is mounted at the distal end of the shaft 18 and can rotate relative to the shaft 18 by virtue of bearing 41. The rotatable member 42 is coupled to the rotation mechanism of FIG. 3 by means of a hollow torque-transmitting member 19 (which may for example be a torque coil or a torque shaft) which passes inside the shaft 18 (see also the inset of FIG. 1). The main drive cable 20 runs inside the torque-transmitting member 19 and is connected to a first linkage 44 of the end effector mechanism.

With the illustrated rotation mechanism, the shaft 18 (at the end of which the end effector is mounted) does not rotate as the user turns the rotary adjustment knob 22. This is due to the shaft 18 being non-rotatably attached to the body part 12 by means of bridging part 24. The shaft 18 also incorporates a fixed hollow axle 23 which passes beneath the bridging part 24 and about which the rotary adjustment knob 22 is rotatably mounted by means of a cylindrical bearing 38.

The abovementioned torque-transmitting member 19 is provided within the hollow shaft 18 and the hollow axle 23. In embodiments in which the shaft 18 is flexible or curved, the torque-transmitting member 19 may be a torque coil. Alternatively, in embodiments in which the shaft 18 is rigid and straight, the torque-transmitting member 19 may be a torque rod (or a torque coil). As shown in FIG. 3, a proximal end of the torque-transmitting member 19 is rigidly attached to the rotary adjustment knob 22, e.g. by means of fixture 11. Further, as shown in FIG. 4a, a distal end of the torque-transmitting member 19 is rigidly attached to the rotatable member 42.

As mentioned above, the main drive cable 20 passes along the hollow centre of the torque-transmitting member 19 and, at the distal end 40 of the shaft 18, is connected to the first linkage 44 of the end effector mechanism.

The first linkage 44 of the end effector mechanism is resiliently biased in a direction away from the body part 12, to apply a restoring force on the drive cable 20 for reversing the action of the actuating member 14. In the case of FIG. 4a, this resilient biasing is provided by a compression spring 46 that acts between the rotatable member 42 and the first linkage 44.

In use, by the surgeon applying finger pressure on the actuating member 14, the drive cable 20 is moved axially, in turn moving the linkage 44 and thus actuating the end effector. More particularly, as the actuating member 14 is pressed towards the body part 12 of the handle, the drive cable 20 is pulled in the direction away from the end effector, toward the body part 12, thus pulling on the linkage 44, actuating the end effector and compressing the compression spring 46. Conversely, as the surgeon removes pressure from the actuating member 14, the drive cable 20 moves in the opposite direction (i.e. in the direction of the end effector), pulled by the restoring force exerted by the compression spring 46 on the linkage 44, and causing (via reverse operation of the pulley mechanism and the rack and pinion mechanism) the actuating member 14 to move away from the body part 12 of the handle.

FIG. 4b is a cross-sectional illustration of an alternative arrangement for spring-biasing the end effector, that may be used if the end effector is not to be rotatable relative to the shaft 18. This may be the case if the end effector is not to be rotatable at all, or if the shaft 18 is rotatable relative to the body part 12 but the end effector is non-rotatable relative to the shaft 18. In all such cases, it will be appreciated that there is no need for a torque-transmitting member 19 or a rotatable member 42 as described above.

Instead, in the case of FIG. 4b, the first linkage of the end effector mechanism is provided by, or attached to, one end of a shaft 21 that is slidably mounted within the distal end of the shaft 18. The main drive cable 20 is attached to the other end of the shaft 21. The shaft 21 has an enlarged portion 25, whereas the shaft 18 has a restricted portion 18 with a central aperture therein. The end of the shaft 21 to which the drive cable 20 is attached is able to pass freely through the central aperture in the restricted portion 18a, whereas the enlarged potion 25 of the shaft 21 is too large to pass through this central aperture.

The shaft 21 (and thus the first linkage of the end effector mechanism) is resiliently biased in a direction away from the body part 12, to apply a restoring force on the drive cable 20 for reversing the action of the actuating member 14. As in the case of FIG. 4a, in the variant of FIG. 4b this resilient biasing is provided by a compression spring 46—in this case acting between the enlarged portion 25 of the shaft 21 and the restricted portion 18a of the shaft 18.

In use, by the surgeon applying finger pressure on the actuating member 14, the drive cable 20 is moved axially, in turn moving the shaft 21 and thus actuating the end effector. More particularly, as the actuating member 14 is pressed towards the body part 12 of the handle, the drive cable 20 is pulled in the direction away from the end effector, toward the body part 12, thus pulling on the shaft 21, actuating the end effector and compressing the compression spring 46. Conversely, as the surgeon removes pressure from the actuating member 14, the drive cable 20 moves in the opposite direction, pulled by the restoring force exerted by the compression spring 46 on the enlarged portion 25 of the shaft 21, and causing (via reverse operation of the pulley mechanism and the rack and pinion mechanism) the actuating member 14 to move away from the body part 12 of the handle.

The principle of providing a compression spring 46 to resiliently bias a linkage 44 or other part of the end effector mechanism in a direction away from the body part 12, and thereby apply a restoring force on the drive cable 20 for reversing the action of the actuating member, is applicable to all the embodiments and variants of the actuator handle described herein.

FIG. 5 is a cross-sectional view of a variant of the embodiment of FIG. 1. In this case, the handle 10′ has a push-button actuating member 14 connected to an alternative force transmission mechanism within the body part 12 of the handle 10′. As before, the push-button actuating member 14 is arranged to be operably moveable by a single finger of the user's hand (the same hand as supports the body part 12), in a linear or near-linear manner between a first (outward) position and a second (inward) position relative to the body part 12, for actuating the end effector. Thus, by gripping the body part 12, the user is able to hold the entirety of the tool still (with the exception of the actuating member 14, that is moved by the single finger), in a fixed position relative to the user's hand. The user's hand also remains still during the actuation process, with the exception of the single finger that moves to operate the actuating member 14.

In more detail, in the variant of FIG. 5, the push-button actuating member 14 is provided with a shaft 16 that is slidably mounted within a guide aperture in the casing of the body part 12. The sliding movement of the shaft 16 may be constrained for example by a fixed pin located within a slot in the shaft 16.

In the variant of FIG. 5, rather than using a rack and pinion mechanism, the shaft is provided with a fixing 17 to which the cable 30 is directly attached. The cable 30 passes around a pulley mechanism (fixed pulley 29, moveable pulley 32, fixed pulley 34, and a further pulley that is not shown in FIG. 5 but corresponds to pulley 36 of FIG. 2) to transmit motive force to the main drive cable 20 and thence to the end effector, in much the same manner as in the embodiment of FIGS. 1 and 2.

FIG. 5 also includes a cutaway 16a showing that a slot may be provided in the shaft 16 for the cable 20 to pass through. (Alternatively, of course, the cable could pass either side of the shaft 16, depending on the planar alignment of the various pulleys.)

FIG. 6 illustrates the actuator handle of FIG. 1 in use, with the user's hand gripping and operating the handle using a precision “pencil” grip between their thumb and middle finger, and with their index finger on the push-button actuating member.

Lever Arm Actuating Member

FIG. 7 is a cross-sectional view of a second embodiment of an actuator handle 50 for a hand-held surgical tool. In this embodiment the handle 50 has a rotary lever arm actuating member 54 connected to a force transmission mechanism within the body part 12 of the handle 50. Similar to the above-described embodiments, the rotary lever arm actuating member 54 is arranged to be operably moveable by a single finger of the user's hand (the same hand as supports the body part 12), in a near-linear manner between a first (outward) position and a second (inward) position relative to the body part 12, for actuating the end effector. Thus, by gripping the body part 12, the user is able to hold the entirety of the tool still (with the exception of the actuating member 54, that is moved by the single finger), in a fixed position relative to the user's hand. The user's hand also remains still during the actuation process, with the exception of the single finger that moves to operate the actuating member 54.

In more detail, in this embodiment, one end (74) of the rotary lever arm actuating member 54 is rotatably mounted within the body part 12 of the handle 50 and incorporates a spool 75 to which cable 30 is windingly attached. The cable 30 passes around a pulley mechanism (moveable pulley 32 and fixed pulley 34) to transmit motive force to the main drive cable 20 and thence to the end effector, in much the same manner as in the embodiment of FIGS. 1 and 2. As illustrated with this embodiment (although the principle applies equally to any of the embodiments described herein), one or more fixed guide wheels 76 may also be provided to guide any of the cables (in this case, drive cable 20) along a desired path.

In this embodiment, the length of the rotary lever arm actuating member 54 (specifically, the distance (d) of the pushed region of the actuating member 54 from the rotatably-mounted end 74) provides an inherent mechanical advantage. As those skilled in the art will appreciate, the mechanical advantage also depends on the radius (r) at which the cable 30 is attached to the spool 75, the mechanical advantage being given by d/r.

Lever Arm Actuating Member with Scott-Russell Linkages

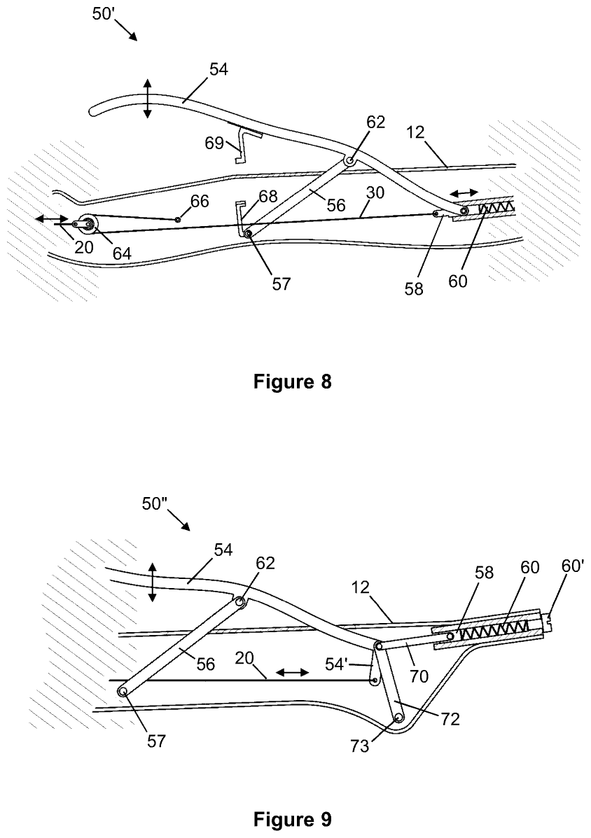

FIG. 8 is a cross-sectional view of a third embodiment of an actuator handle 50′ for a hand-held surgical tool. As with the first and second embodiments (and their variants) as described above, the handle 50′ comprises a body part 12 adapted to be gripped by a surgeon's hand in use, and which has an end effector mounted or mountable thereon. In this case, though, the handle 50′ comprises a lever arm actuating member 54 that forms part of a Scott-Russell linkage mechanism. A cable and pulley arrangement is connected to the Scott-Russell linkage mechanism to form a force transmission (and force magnification) mechanism for actuating the end effector. As with the above-described embodiments, the lever arm actuating member 54 is arranged to be operably moveable by a single finger of the user's hand (the same hand as supports the body part 12), in a linear or near-linear manner between a first (outward) position and a second (inward) position relative to the body part 12, for actuating the end effector. Thus, by gripping the body part 12, the user is able to hold the entirety of the tool still (with the exception of the actuating member 54, that is moved by the single finger), in a fixed position relative to the user's hand.

The user's hand also remains still during the actuation process, with the exception of the single finger that moves to operate the actuating member 54.

In more detail, in this embodiment the Scott-Russell linkage mechanism is made up of the lever arm actuating member 54, linkage 56, and sliding member 58. One end of linkage 56 is pivotally mounted about pivot point 57, whereas the other end of linkage 56 is pivotally connected to a pivot point 62 along the length of the lever arm actuating member 54. The inward end of the lever arm actuating member 54 is pivotally connected to the sliding member 58.

To the sliding member 58 is attached a cable 30 that passes around a movable pulley 64 and is fastened to fixed point 66. The main drive cable 20 is also attached to the moveable pulley 64, in the manner as illustrated, to provide additional mechanical advantage (with the Scott-Russell linkage mechanism itself providing some mechanical advantage of its own). By the surgeon pushing the lever arm actuating member 54, the Scott-Russell linkage mechanism pulls the cable 30, which transmits motive force to the main drive cable 20 and thence to the end effector.

Naturally, if appropriate, the Scott-Russell linkage mechanism can also be used alone, without any pulleys to magnify the force further.

It will be appreciated that the Scott-Russell linkage mechanism provides reciprocating motion of a connection point within said linkage mechanism in response to movement of the actuating member 54 between the first and second positions, to which connection point the end effector is coupled by means of the cable 30 or other coupling components. The Scott-Russell linkage mechanism also allows for substantially straight line motion of the actuating finger between the first (outward) and second (inward) positions.

In the illustrated embodiment a locking mechanism, for locking the lever arm actuating member 54 in the second (inward) position, is provided by reversibly-engageable locking members 68 and 69. These locking members 68 and 69 mutually engage as they are brought together, and have the effect of locking the end effector in its actuated state. The locking members 68 and 69 can subsequently be disengaged by pushing again on the lever arm actuating member 54, thereby unlocking the end effector from its actuated state.

A compression spring 60 is optionally provided for the sliding member 58 to act against, to provide a degree of resistance to the motion of the lever arm actuating member 54 and the Scott-Russell linkage mechanism more generally. Such a compression spring 60 may be adjustable, to adjust the degree of resistance it provides. For example, as shown in FIG. 9, the compression spring 60 may be adjusted by means of adjustment screw 60′. It should however be appreciated that a degree of resistance (along with the generation of a restoring force on the drive cable 20) will also be provided by the compression spring 46 at the end effector end of the tool, and so compression spring 60 should not be considered as essential. Alternatively, if, instead of a cable, a rigid linkage/rod is attached to the sliding member 58 and runs directly to the end effector, then only restoring spring 60 would be required.

FIG. 9 is a cross-sectional view of a variant of the actuator handle of FIG. 8, with the lever arm forming part of a modified Scott-Russell linkage mechanism to which the drive cable 20 (that transmits motive force from the lever arm actuating member 54 to the end effector) is directly connected.

In more detail, in this variant the lever arm actuating member 54 is provided with a further strut 54′ at substantially 90° from the preceding part of the lever arm actuating member 54. The drive cable 20 is connected to the end of the 90° strut 54′. Additional linkages 70 and 72 are also provided. A first end of linkage 70 is pivotally connected to the 90° point between the lever arm actuating member 54 and the further strut 54′. The other end of linkage 7 is pivotally connected to the sliding member 58. Meanwhile, one end of linkage 72 is pivotally mounted about pivot point 73, whereas the other end of linkage 72 is pivotally connected to the same place as the first end of linkage 70.

By the surgeon pushing the lever arm actuating member 54, the Scott-Russell linkage mechanism pulls the cable 30, which transmits motive force to the main drive cable 20 and thence to the end effector.

In a variant of this embodiment, it is possible to omit the linkage 70 and the sliding member 58. Instead of these components, a simple spring may be provided that is compressed when linkage 72 rotates backwards. Additionally, or alternatively, such a spring could be placed under linkage 56 or linkage 54/54′, or could be a torsion spring at any pivot point or joint. Such a spring will thus provide a restoring force to return the actuating member 54 to the first (outward) position.

Compliant Scott-Russell Linkage Mechanism

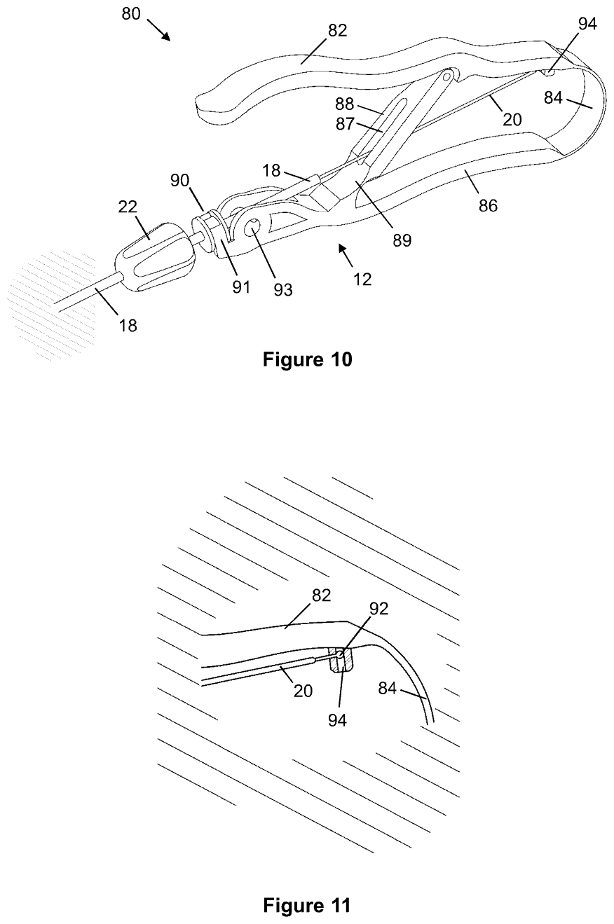

FIGS. 10, 12a, 12b and 13, and FIGS. 15a-15c (in use), illustrate a fourth embodiment of an actuator handle 80 for a hand-held surgical tool. In this embodiment the handle 80 has a lever arm actuating member 82 that forms part of a compliant Scott-Russell linkage mechanism to which the drive cable (or a rigid rod) 20 is connected to form a force transmission mechanism for actuating the end effector. At least a principal part 86 of the Scott-Russell linkage mechanism forms the body part 12 of the handle 80. As with the above-described embodiments, the lever arm actuating member 82 is arranged to be operably moveable by a single finger of the user's hand (the same hand as supports the body part 12), in a linear or near-linear manner between a first (outward) position and a second (inward) position relative to the body part 12, for actuating the end effector.

The overall Scott-Russell linkage mechanism is configured such that the lever arm actuating member 82 has greater ability to move (relative to the rest of the tool) than any of the other parts of the linkage mechanism. By gripping the body part 12, the user is able to hold the entirety of the tool still (with the exception of the actuating member 82, that is moved by the single finger), in a fixed position relative to the user's hand. The user's hand also remains still during the actuation process, with the exception of the single finger that moves to operate the actuating member 82. The inventors arrived at the compliant Scott-Russell linkage mechanism of this embodiment by adapting (simplifying) the mechanisms of FIGS. 8 and 9 whilst substantially preserving the geometrical and mechanical principles therein.

Notably, the illustrated actuator handle 80 is a skeletal structure, wherein at least the principal part 86 of the Scott-Russell linkage mechanism forms a grippable part of the body part 12. That is to say, no additional casing is provided around the members of the Scott-Russell mechanism, and at least the principal part 86 of the Scott-Russell linkage mechanism is directly gripped by the user's hand in use. This enables a number of advantages to be realised, including: reduced weight, reduced number of parts, a potential saving in cost, potentially easier sterilization (by having fewer places in which organic matter can “hide”), and reduced instances of wear (by reducing the number of joints and sliding bearing surfaces). Further advantages include the compliant design allowing for higher energy efficiency (no/less friction), and less (or zero) “slack” in the mechanism (as caused commonly by joint tolerances) leading to improved tactile feedback. Finally, the compliant design allows the handle to remain lighter and to provide a more compact finger gripping, due to not requiring a separate body casing. The above notwithstanding, a variant of the handle 80 may be envisaged in which a casing is provided (entirely or partially) around the linkages of the body part 12.

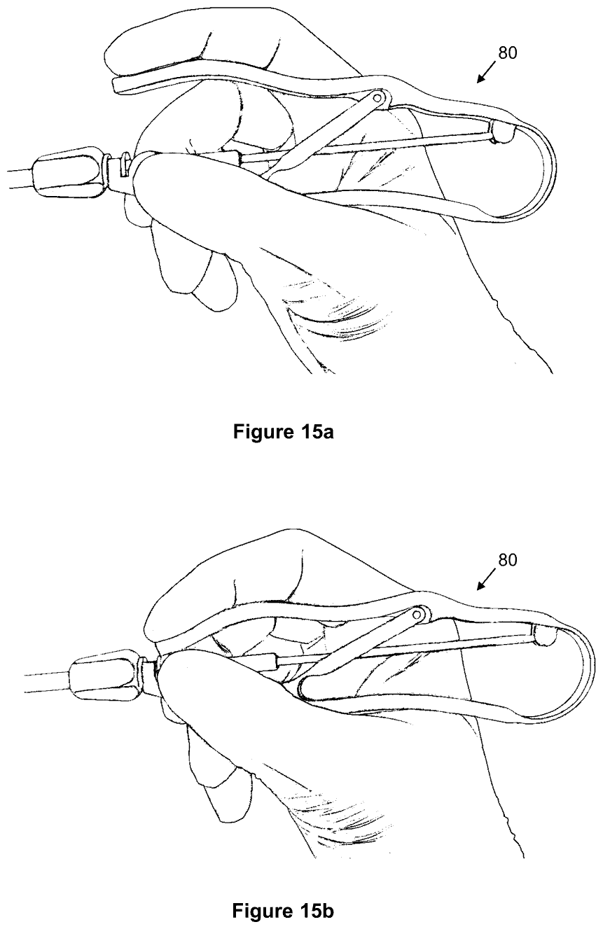

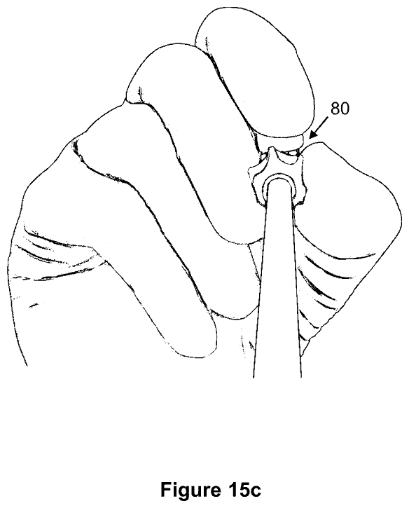

In more detail, with the actuator handle 80, the principal part 86 of the Scott-Russell linkage mechanism comprises guide regions 93 in which to locate the thumb and middle finger of the user's hand in use. The guide regions 93 may for example be holes (as illustrated), or depressions or ridges, or some other tactile feature to aid the user with respect to positioning their thumb and middle finger, and/or to enhance the user's grip. The lever arm actuating member 82 is arranged to be operated by the first finger of the user's same hand, as shown in FIGS. 15a and 15b.

As illustrated, the actuator handle 80 is provided with an optional rotation mechanism, in this case comprising a rotary adjustment knob 22 (to which, in this example, the shaft 18 is rigidly attached) and a rotary bearing 90 mounted by a support member 91. By rotating the adjustment knob 22 (e.g. by slightly moving their middle finger or ring (third) finger to reach the knob 22) the surgeon can adjust the orientation of the shaft 18 and thence the end effector. Such finger-operated potentially—360° axial rotation of the distal end of the tool advantageously avoids the need for the surgeon to rotate their own wrist/arm/body.

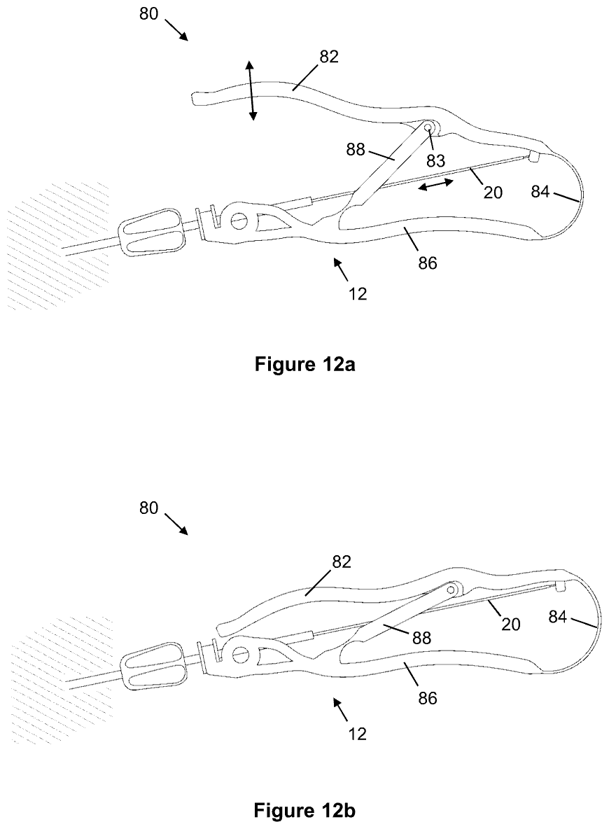

As shown in FIGS. 10 and 11, the drive cable or rod 20 may conveniently be attached to a connection point at the pivot end of the actuating member 82, for example by means of a ball and socket coupling. More particularly, as illustrated, a slot-like socket 94 is formed at the pivot end of the actuating member 82, and a ball 92 is formed at the end of the cable or rod 20. The ball 92 is rotatable (and preferably also slidable) within the slot-like socket 94. This allows pivoting and rotation of the end of the cable or rod 20 relative to the handle, and also longitudinal movement of the end of the cable or rod 20 within the socket, which is beneficial to allow for changes in the angle and position of the cable or rod relative to the handle during operation, e.g. when the actuating member is moved from the first position (as in FIG. 12a) to the second position (as in FIG. 12b).

The members of the compliant Scott-Russell linkage mechanism include the principal part 86, the actuating member 82, a first intermediate linkage 84 and a second intermediate linkage 88. As shown, the second intermediate linkage 88 may include an aperture 87 through which the drive cable or rod 20 passes, en route to the end effector.

As shown in FIGS. 10, 12a, 12b and 13e the actuating member 82 may advantageously be formed in a compliant unitary manner with the principal part 86. Preferably, as shown, this unitary formation may be achieved by the first intermediate linkage 84 also being formed in a compliant unitary manner with both the actuating member 82 and the principal part 86—for example, made of surgical steel, titanium, nitinol, or another suitable material.

Particularly preferably, as shown, the first intermediate linkage 84 is formed of thinner material than both the actuating member 82 and the principal part 86, to enable preferential flexure of the first intermediate linkage 84 relative to both the actuating member 82 and the principal part 86.

Preferably, as shown, the second intermediate linkage 88 is also formed in a compliant unitary manner with the principal part 86 (although alternatively the second intermediate linkage 88 may be formed in a compliant unitary manner with the actuating member 82).

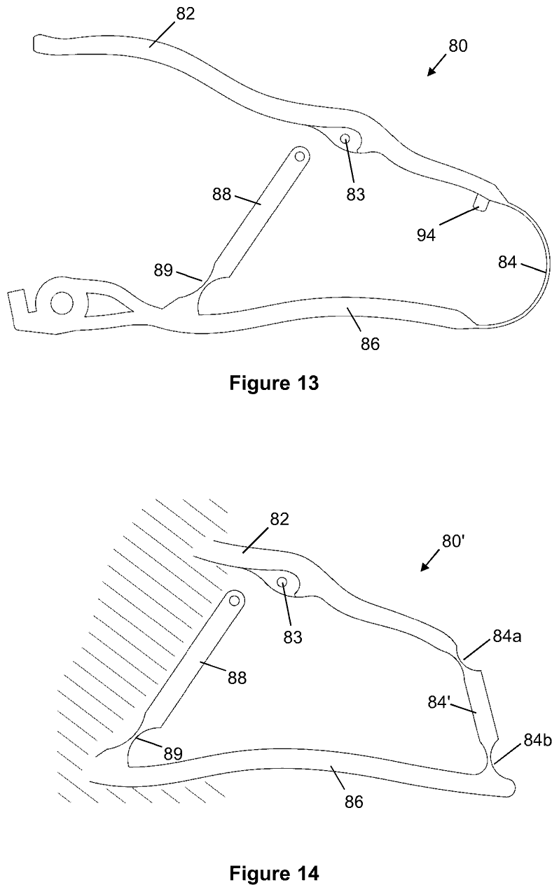

With reference to FIG. 13, initially, during manufacture, the second intermediate linkage 88 is not connected to the actuating member 82, but is connected to the principal part 86 by means of a flexible waisted region 89. Subsequently, during manufacture, the compliant Scott-Russell linkage mechanism is flexed to enable the internal end of the second intermediate linkage 88 to be pivotally connected to a pivot point 83 partway along the actuating member 82. In such a manner the compliant Scott-Russell linkage mechanism is inherently put in tension—i.e. is pretensioned.

Such pretensioning of the compliant Scott-Russell linkage mechanism provides an inherent level of resistance when, in use, the user pushes the actuating member 82 from the first position (FIG. 12a) to the second position (FIG. 12b) to actuate the end effector. Such pretensioning also provides an inherent restoring force to return the actuating member 82 to the first position (FIG. 12a). Moreover, as a consequence of such pretensioning, no restoring spring need be provided at the end effector when a rigid rod 20 is used rather than a cable, with the pretensioning reversing the operation of the end effector as the user reduces the pressure exerted by their finger on the actuating member 82.

If, on the other hand, a cable 20 is used to drive the end effector, then the end effector will need to be biased to prevent cable slack. In such cases, the pretensioning provided by the compliant Scott-Russell linkage mechanism is still beneficial, as the end effector biasing spring need only be responsible for providing cable tension, and not for restoring the actuating member 82 to the first position.

With reference to FIG. 14, in a variant 80′ of the actuator handle 80, rather than the first intermediate linkage 84 being formed entirely of thinner material, the first intermediate linkage 84′ may alternatively be made of thicker material, with a flexible waisted region 84a joining the first intermediate linkage 84′ to the actuating member 82, and a flexible waisted region 84b joining the first intermediate linkage 84′ to the principal part 86. Accordingly, the mechanism of FIG. 14 is less compliant than that of handle 80 as described above.

In passing, it may be noted that variant 80′ was developed as an early prototype of the aforementioned compliant actuator handle 80. The inventors developed the compliant actuator handle 80 after first designing the variant 80′ and then, after further design work, realising that the second intermediate linkage 84′ and the waisted regions 84a and 84b may be converted into a single continuous hinge-like linkage 84 as shown in the earlier figures, resulting in the compliant handle 80.

FIGS. 15a, 15b and 15c show the compliant handle 80 in use. It can be seen that the body of the handle is securely (and compactly) gripped in a precision “pencil” grip, between the user's thumb and middle finger, with their first finger on the actuating member 82 for operating the end effector in a mechanically isolated manner that does not disturb the surgeon's steady holding of the overall tool. Indeed, as well as single finger actuation keeping the end effector more stable, the use of a precision grip (which biomechanically is advantageous for small and precise hand movements) also enhances the overall stability of the tool. Moreover, from the end-on view in FIG. 15 it will be appreciated that the compact form of the handle 80 gives the surgeon excellent visibility of the surgical site and the end effector when looking along the length of the shaft, even in narrow anatomical corridors.

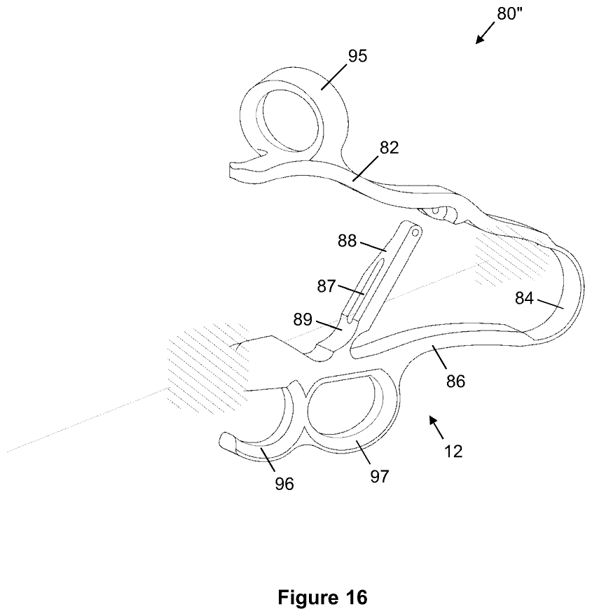

FIG. 16 illustrates an actuator handle 80″ that is a variant of the actuator handle 50 of FIG. 10, with the body part 12 and/or the actuating member 82 comprising one or more finger rings (95, 97) or finger tangs (e.g. 96) in which to locate one or more fingers or the thumb of the user's hand in use.

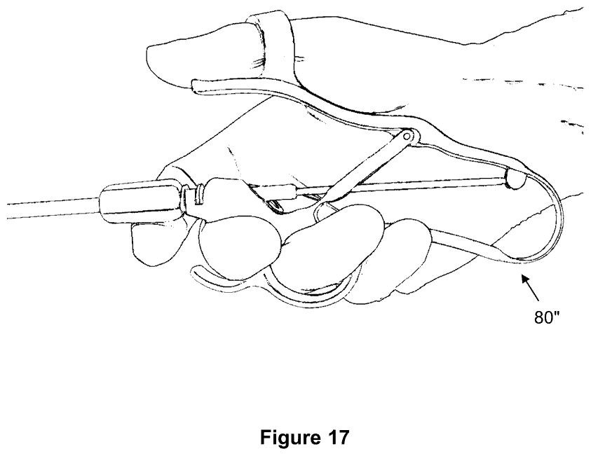

In relation to this, FIG. 17 illustrates the actuator handle of FIG. 16 in use. This shows the user's hand gripping and operating the handle using an ergonomic “rope” grip, with their thumb located in the finger ring 95 on the actuating member 82, their ring finger located in the finger ring 97 on the body part, and their middle finger located in the finger tang 96 on the body part. Accordingly, their first finger is free to operate the adjustment knob 22 (as shown in FIG. 10).

Of note, the arrangement of the handle mechanism could be inverted to enable a “pistol” grip, by providing increased angulation between the actuator handle and the main axis of the instrument. In such a case the actuating member 82 could instead be triggered by movement of the index, middle and ring (third) fingers, or by the main body of the base of the thumb and adjoining palm. The degree of angulation could be set to suit individual surgeon preference.

The actuator handles described herein are intended to be ergonomically designed to conform closely to the surgeon's hand. To this end, any of the handles described herein may be custom designed to specifically conform to a given individual's hand shape. In this regard, FIGS. 15a, 15b and 17 show the close conformance that can be achieved between the respective actuator handles and the surgeon's hand, through the actuator handles being custom designed. In turn, this results in comfortable and precise operation of the tool by the surgeon.

Surgical Clips

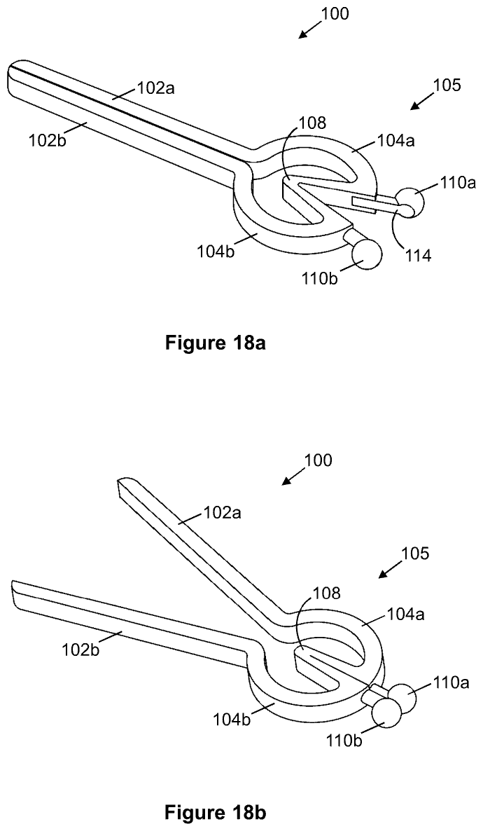

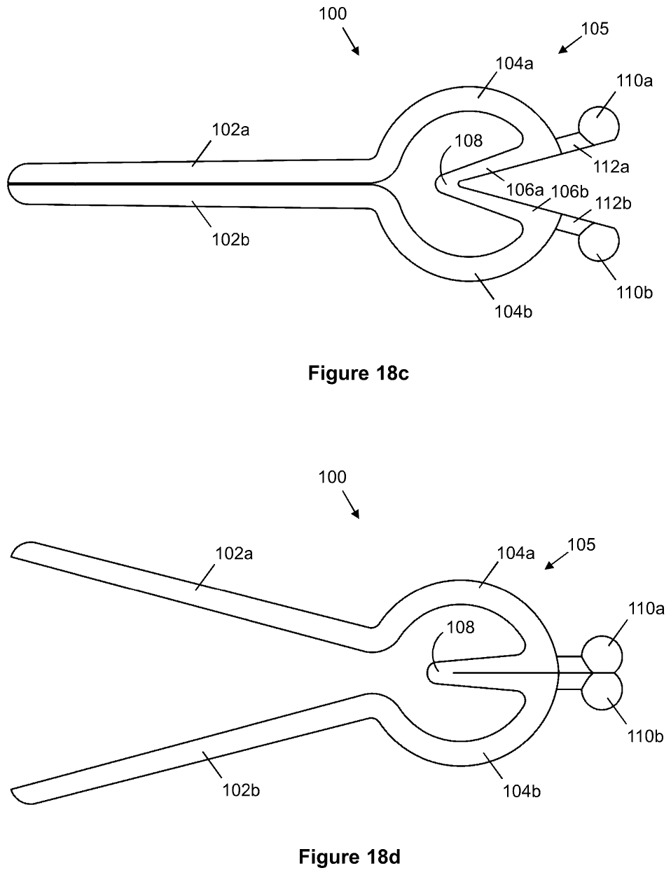

By way of an initial overview, and with reference initially to the embodiment of FIGS. 18a-18d, the present work also provides a surgical clip 100, for example for use in aneurysm clipping, the clip 100 comprising first and second clipping arms 102a, 102b that are mutually openable and closable, and first and second engagement fixtures 110a, 110b respectively coupled to the first and second clipping arms 102a, 102b, by means of which the first and second clipping arms 102a, 102b may be opened and closed.

By means of the inherent resilience of the material from which the clip 100 is made (as is the case with the embodiment of FIGS. 18a-18d), or by means of an attached spring component, the first and second clipping arms 102a, 102b are biased into a closed configuration (so that, once deployed, e.g. around an aneurysm, the clips will maintain their clipping function indefinitely).

For example, the clips of the present work may be made of surgical steel, titanium, nitinol, or another suitable material. Robust resilient polymer materials, or polymer matrix composite materials, may also be used. Advantageously, such polymer materials may be transparent, thereby enabling at least part of the clip to be transparent, which in turn enables the surgeon to see the clipped anatomical feature (e.g. vessel) through the clip.

If desired, the clips may be multi-material, for example with the first and second clipping arms 102a, 102b being made of a different material than the biasing part.

With reference in passing to the example of FIG. 20, the first and second engagement fixtures 110a, 110b are shaped and configured such that first and second gripping arms (e.g. 162a, 162b in FIG. 20) of a complementary surgical clip deployment tool (of which a distal end 40 is shown in FIG. 20) can grip the clip 100 and open the clipping arms 102a, 102b by applying a lateral force to the first and second engagement fixtures 110a, 110b. In such a manner the clip 100 can be moved to the surgical site and opened to enable it to be located around the anatomical feature or defect to be clipped.

In the illustrated embodiments the lateral force required to open the clipping arms 102a, 102b is an inward gripping force, to urge the first and second engagement fixtures 110a, 110b towards one another, and to overcome the abovementioned biasing of the clip into the closed position. However, it will be appreciated that alternative clip designs (not illustrated) may be realised in which the lateral force required to grip and open the clipping arms is an outward force, so as to urge the first and second engagement fixtures apart from one another (and still overcoming the abovementioned biasing of the clip into the closed position).

It will be appreciated that some clip deployment tools may enable different levels of lateral force to be applied to the first and second engagement fixtures 110a, 110b. In particular, a first (relatively low) level of lateral force may be applied that is sufficient to grip the clip 100 and enable it to be moved into position at the surgical site. Then, under the surgeon's control, a second (higher) level of lateral force may be applied, that is sufficient to overcome the abovementioned biasing of the clip and cause the first and second clipping arms 102a, 102b to open. The extent to which the clipping arms are opened will depend on the level of lateral force applied, which may be fully variable, under the surgeon's control.

Of particular note, in the example illustrated in FIGS. 18a-18d, each of the first and second engagement fixtures 110a, 110b comprises at least part of a ball, with which each of the arms 162a, 162b of a complementary surgical clip deployment tool can engage in a substantially ball-and-socket manner, as shown for example in FIGS. and 22. More particularly, in this case, each of the arms 162a, 162b of the complementary surgical clip deployment tool is provided with a respective loop- (or cup-) shaped tip 164a, 164b, shaped to provide the “socket” role in the ball-and-socket relationship with the first and second engagement fixtures 110a, 110b of the clip 100.

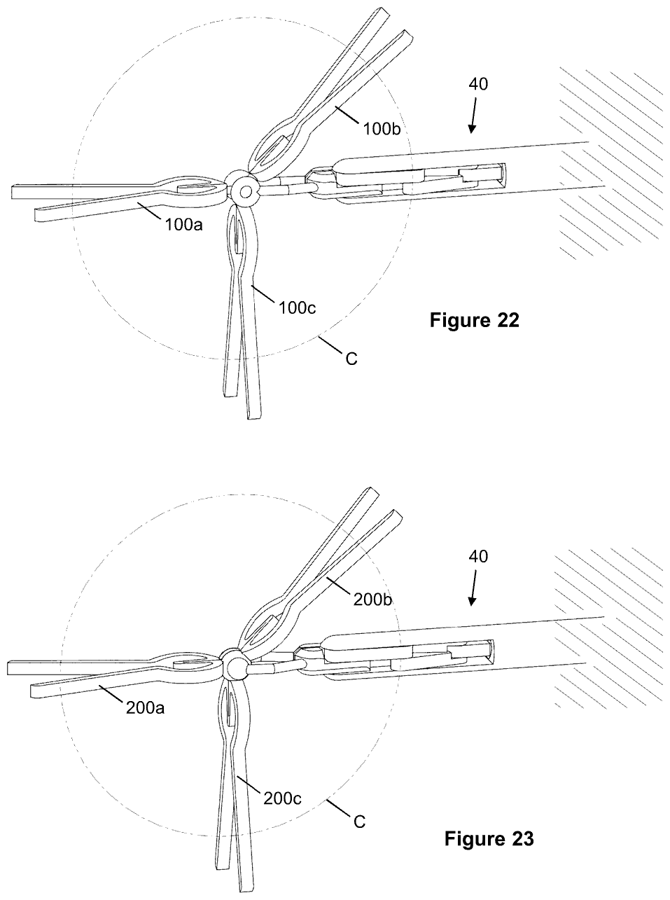

In the illustrated engagement fixtures 110a, 110b the shape of the “ball” is at least partially spherical, and the sockets provided by the tool tips 164a, 164b are circular, which is advantageous for the reasons discussed below in relation to FIG. 22, in terms of providing full flexibility of rotation of the angle of the clip 100 relative to the tool.

More specifically, in the example of FIGS. 18a-18d, the “balls” of the first and second engagement fixtures 110a, 110b are not completely spherical. The inward facing side of each ball is flattened to allow the two “balls” to get closer together and open the clip as wide as possible. Thus, it will be appreciated that the “ball” surface is only necessary so far as it engages the applicator. Beyond that, the first and second engagement fixtures 110a, 110b may be trimmed flat to increase the opening ability of the clip.

In alternative embodiments the shape of the “ball” of each engagement fixture 110a, 110b may at least partially be a geometric solid (other than a sphere) having rotational symmetry, to be gripped by tool tips 164a, 164b that have a corresponding geometric-shaped socket having rotational symmetry. For example, a triangular-pyramid shaped “ball” may be gripped by tool tips that are triangular in shape; a cubic “ball” may be gripped by tool tips that are square in shape; and more complex geometric solid “ball” shapes may be gripped by tool tips that are hexagonal or octagonal in shape. Such configurations still permit rotation of the angle of the clip 100 relative to the tool, but limited to certain angles as defined by the rotational symmetry of the shapes of the “ball” and socket. Such angular limitation facilitates ‘locking’ of the clip at a preferred angle in the applicator, optimised for a predetermined surgical trajectory.

In more detail, the clip 100 further comprises a hinge portion 105 by means of which the clipping arms 102a, 102b are openable and closable. The hinge portion 105 also provides the abovementioned biasing of the clip into the closed position.

In this embodiment (and many of the others described or illustrated herein), the clipping arms 102a, 102b and the hinge portion 105 are coplanar and of unitary form, preferably manufactured from (or as) a single piece of material.

In the illustrated example, the hinge portion 105 comprises first and second outer members 104a, 104b that are respectively attached to the first and second clipping arms 102a, 102b. The first and second outer members 104a, 104b are also respectively attached to the first and second engagement fixtures 110a, 110b. It will be appreciated that, in this example, the first and second engagement fixtures 110a, 110b are on the opposite side of the hinge portion 105 from the first and second clipping arms 102, 102b.

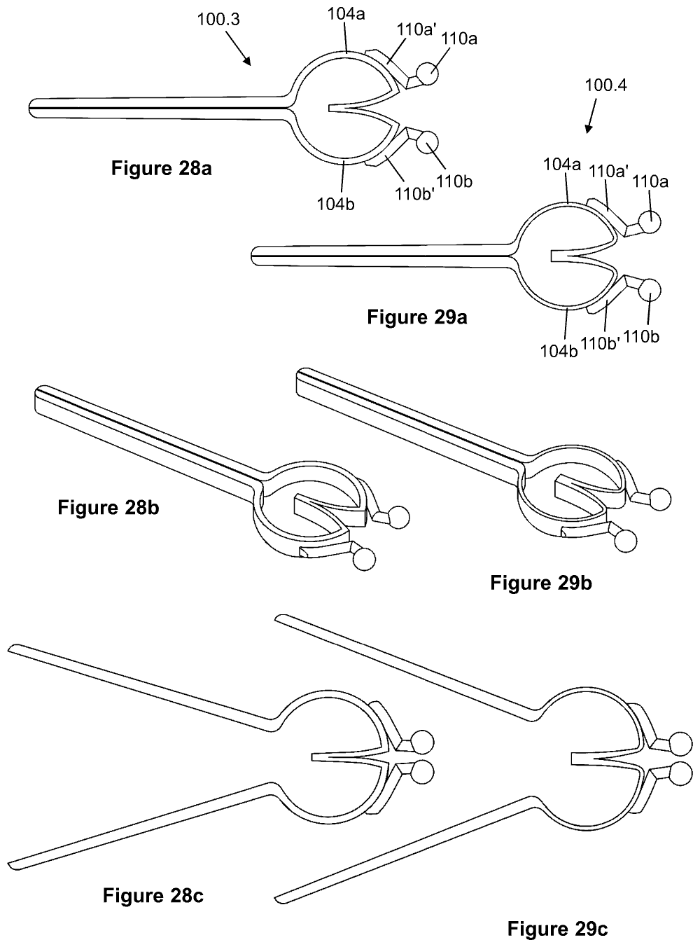

Further, within the hinge portion 105, a resilient hinge member 108 is provided, between, and coupled to, the first and second outer members 104a, 104b. In this example the resilient hinge member 108 is substantially V-shaped (comprising arms 106a, 106b that together form the “V”). The resilient hinge member 108 provides the abovementioned biasing of the clip into the closed position, and enables the clip to resiliently flex between closed and open configurations. (In passing, it should be noted that not all the resilient biasing and flexural ability of the clip is necessarily due to the hinge member. In other embodiments, e.g. as shown in FIGS. 28a-28c and 29a-29c, further resilient biasing and flexural ability of the clip may be provided by the first and second outer members 104a, 104b.)

By means of the resilient hinge member 108 the first and second outer members 104a, 104b, and thence the first and second clipping arms 102a, 102b, can move upon application of lateral force to the first and second engagement fixtures 110a, 110b by the clip deployment tool. As shown in FIGS. 18b and 18d, a state of maximal opening of the clip 100 is achieved when sufficient lateral force is applied to the first and second engagement fixtures 110a, 110b to close the V-shape of the hinge member 108 (or to bring the engagement fixtures 110a, 110b into contact with one another).

As shown in FIG. 18a, to safeguard against the possibility of the first and second engagement fixtures 110a, 110b being sheared off the first and second outer members 104a, 104b upon application of lateral force, a reinforcing member 114 may be incorporated in each of the first and second engagement fixtures 110a, 110b, extending into the first and second outer members 104a, 104b (and potentially further extending into the arms 106a, 106b of the V-shaped resilient hinge member 108).

In the illustrated clip 100, the hinge portion 105 and the first and second clipping arms 102a, 102b are coplanar and of unitary compliant form. Advantageously they may be formed as (or from) a single piece of material. However, this need not necessarily be the case, as they may alternatively be made from separate materials or separate components that are joined (e.g. fused) together during manufacture. For example, the hinge portion 105 (and the resilient hinge member 108 in particular) may be made of a different material from the first and second clipping arms 102a, 102b.

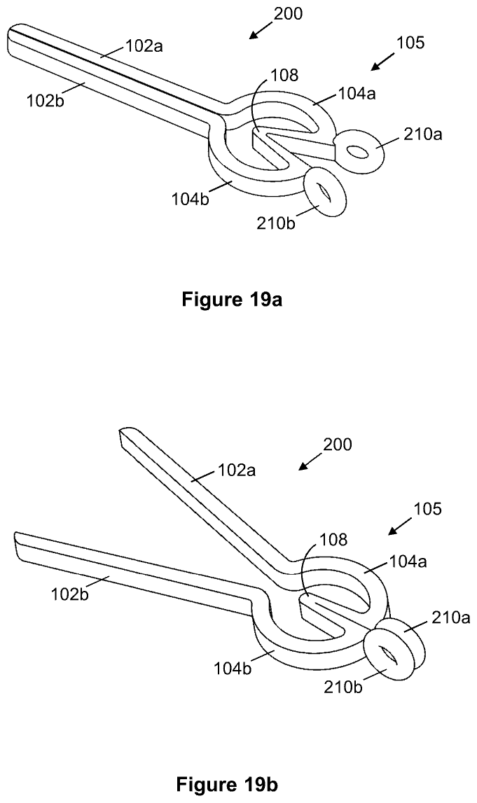

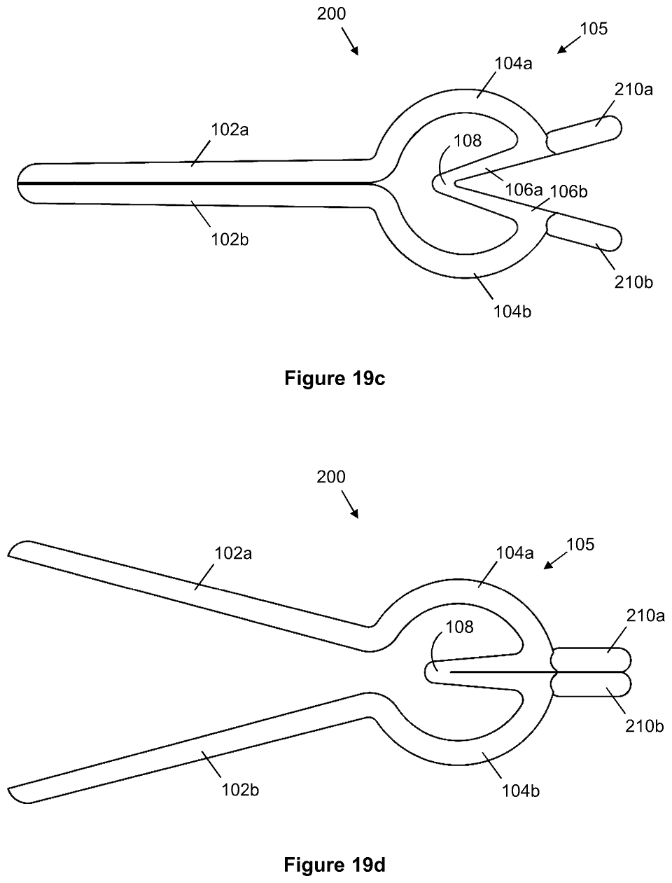

FIGS. 19a-19d illustrate, in closed and open configurations respectively, a variant of the clip of FIGS. 18a-18d. In the clip 200 of FIGS. 19a-19d, each of the first and second engagement fixtures 210a, 210b comprises at least part of a loop (or cup), with which each of the arms 262a, 262b of a complementary surgical clip deployment tool can engage in a substantially ball-and-socket manner, as shown for example in FIGS. 21 and 23. More particularly, in this case, each of the arms 262a, 262b of the complementary surgical clip deployment tool is provided with a respective ball-shaped tip 264a, 264b, shaped to provide the “ball” role in the ball-and-socket relationship with the first and second engagement fixtures 210a, 210b of the clip 200.

In the illustrated engagement fixtures 210a, 210b the shape of the loop (or cup) is at least partially circular, and the tool tips 264a, 264b are at least partially spherical, which is advantageous for the reasons discussed below in relation to FIG. 23, in terms of providing full flexibility of rotation of the angle of the clip 200 relative to the tool.

However, in alternative embodiments the shape of the loop (or cup) of each engagement fixture 210a, 210b may at least partially be a geometric shape (other than a circle) having rotational symmetry, to be gripped by tool tips 264a, 264b that have a corresponding geometric solid shape having rotational symmetry. For example, a triangular loop may be gripped by tool tips that are triangular pyramidal in shape; a square loop may be gripped by tool tips that are cubic in shape; and hexagonal or octagonal loops may be gripped by tool tips having more complex geometric solid shapes.

In all other respects, the structure and operation of clip 200 is the same as that of clip 100 as described above.

FIG. 20 illustrates the clip 100 of FIGS. 18a-18d being deployed by a complementary clip deployment tool—in this example, an end effector as may potentially be actuated by the actuator handle of any of FIGS. 1-17.

The distal end 40 of the end effector comprises a body (e.g. rotatable member 42 as described above) in which a first linkage 44 is able to move in a reciprocating manner, driven by a drive cable or rod 20 as described above, in response to actuation of the end effector by the user.

The first linkage 44 is, in turn, connected to a so-called “scissor mechanism comprising a first pair of linkages 158a, 158b that are respectively connected to a second pair of linkages 160a, 160b. Linkages 158a, 158b are pivotally connected to the first linkage 44. Linkages 160a, 160b are pivoted about point 170 on the body 42 of the end effector, and extend forward of the pivot point 170. In turn, gripping arm 162a is connected to linkage 160a, and gripping arm 162b is connected to linkage 160b. Gripping arm 162a is terminated by gripping tip 164a, whereas gripping arm 162b is terminated by gripping tip 164b (the gripping tips 164a, 164b being loop-shaped in this case).

Depending on the state of actuation of the tool, the configuration of the gripping arms 162a, 162b and gripping tips 164a, 164b may be closed (as in FIG. 20(a)) or open (as in FIG. 20(b)). When using the actuator handles of the present work, at rest the gripping arms 162a, 162b and gripping tips 164a, 164b naturally adopt an open configuration, as in FIG. 20b, due to the first linkage 44 of the end effector mechanism being biased into a forward position, e.g. by the restorative action of the compression spring 46 as described above.

Then, upon actuation of the handle by the surgeon, e.g. depressing an above-described actuating member from the first (outward) position towards the second (inward) position, the first linkage 44 is pulled by the drive cable or rod 20, away from the distal end of the tool, towards the body part of the tool. This has the effect of pulling the gripping arms 162a, 162b and gripping tips 164a, 164b closer together, causing them, when suitably aligned with the clip, to exert a lateral force on the first and second engagement fixtures 110a, 110b of the clip 100. Initially, such lateral force may only be sufficient to grip the clip 100 but not cause the clipping arms 102a, 102b to open. This may be appropriate for moving the clip to the surgical site.

Upon further actuation of the handle by the surgeon (e.g. depressing the above-described actuating member further towards the second (inward) position), the gripping arms 162a, 162b and gripping tips 164a, 164 will exert a greater lateral force on the first and second engagement fixtures 110a, 110b of the clip 100, causing the clipping arms 102a, 102b to open.

Subsequently, as the surgeon releases the actuating member, the lateral force exerted by the gripping arms 162a, 162b and gripping tips 164a, 164 on the first and second engagement fixtures 110a, 110b of the clip 100 will decrease, causing the clipping arms 102a, 102b to close.

In passing, it will be appreciated that the force magnification capabilities of the above-described actuator handles enables the clip 100 to have a relatively high level of inherent stiffness (thereby enabling it to perform its clipping function reliably over time) whilst being able to be opened and closed in a controlled manner by operation of the actuator handle by the surgeon.

FIG. 21 is similar to FIG. 20, and illustrates the clip 200 of FIGS. 19a-19d being deployed by a clip deployment tool. The details of the distal end 40 of the end effector, and its manner of operation, are as described above with respect to FIG. 20. However, the gripping arms 162a, 162b of the tool in FIG. 21 terminate in ball-shaped gripping tips 264a, 264b (the gripping tips 264a, 264b being spherical in this case), for engaging with the loop- (or cup-) shaped engagement fixtures 210a, 210b of the clip 200.

By virtue of the rotational symmetry of the ball-and-socket manner of engagement between each engagement fixture of the clip, and each gripping tip of the tool, each clip may be held in a variety of angular positions by the clip deployment tool. This is illustrated in FIGS. 22 and 23.

More particularly, FIG. 22 illustrates superimposed examples of angular positions in which the clip 100 of FIGS. 18a-18d may be gripped relative to the distal end 40 of the clip deployment tool. Clip 100a represents the clip 100 being held in a “straight ahead” angular position, clip 100b represents the clip 100 being held in a “doubled-back” angular position, and clip 100c represents the clip 100 being held in an intermediate right-angled position. It will of course be appreciated that, with spherical engagement fixtures 110a, 110b on the clip 100, and circular gripping tips 164a, 164b on the tool, an essentially infinite number of angular positions are available in which to hold the clip 100 relative to the tool, as indicated by the circle C.

Similarly, FIG. 23 illustrates superimposed examples of angular positions in which the clip 200 of FIGS. 19a-19d may be gripped relative to the distal end 40 of the clip deployment tool. As above, clip 200a represents the clip 200 being held in a “straight ahead” angular position, clip 200b represents the clip 200 being held in a “doubled-back” angular position, and clip 200c represents the clip 200 being held in an intermediate right-angled position. It will again be appreciated that, with circular engagement fixtures 210a, 210b on the clip 200, and spherical gripping tips 264a, 264b on the tool, an essentially infinite number of angular positions are available in which to hold the clip 200 relative to the tool, as indicated by the circle C.

The same principles (of enabling the clip to be gripped in a variety of angular positions relative to the tool) apply to the other clips described herein.

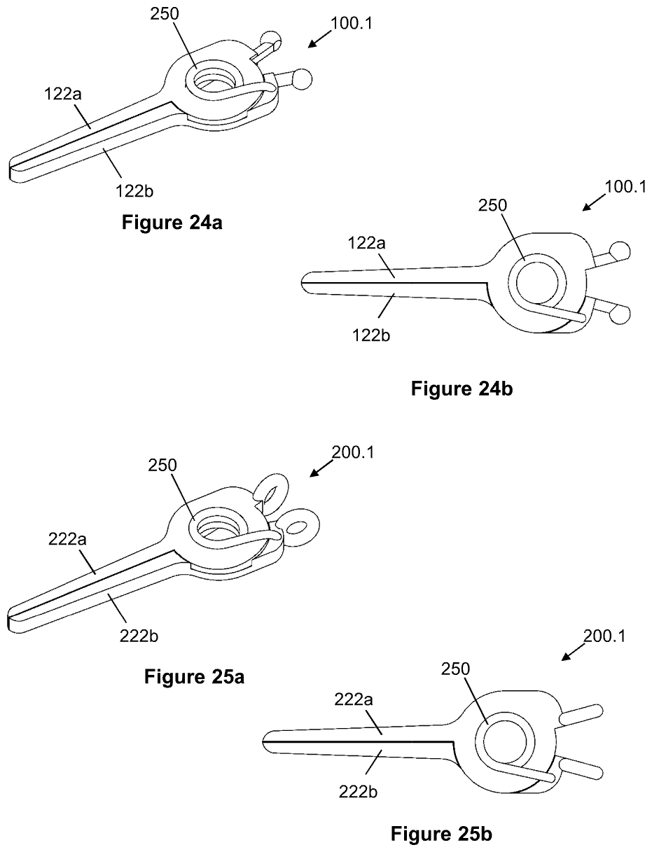

As mentioned above, the biasing means by which the first and second clipping arms are biased into a closed configuration may be provided by a separate spring component, instead of the inherent resilience of the material from which the clip is made. By way of example, FIGS. 24a and 24b illustrate, in perspective and plan views respectively, an alternative configuration of a surgical clip 100.1 comprising first and second (initially separate) principal components 122a, 122b and a separate biasing spring 250 to which the first and second principal components 122a, 122b are both attached during manufacture of the clip. It will be appreciated that the first principal component 122a comprises one of the clipping arms and part of the hinge portion of the clip, whereas the second principal component 122b comprises the other of the clipping arms and a further part of the hinge portion of the clip. The principal components 122a, 122b are shaped and configured such that, once assembled to form the clip, the two clipping arms are coplanar, i.e. side by side, whereas the two parts of the hinge portion are on top of one another and connected together by the spring 250. The spring 250 also serves as a pivot pin for the clipping arms. Each principal component is also provided with a respective engagement fixture (in this case, ball type) by which the clip 100.1 may be gripped and opened using a complementary clip deployment tool, e.g. as outlined above.

Similarly, FIGS. 25a and 25b illustrate, in perspective and plan views respectively, another alternative configuration of a surgical clip 200.1 comprising first and second (initially separate) principal components 222a, 222b and a separate biasing spring 250 to which the first and second principal components 222a, 222b are both attached during manufacture of the clip. It will again be appreciated that the first principal component 222a comprises one of the clipping arms and part of the hinge portion of the clip, whereas the second principal component 222b comprises the other of the clipping arms and a further part of the hinge portion of the clip. The principal components 222a, 222b are shaped and configured such that, once assembled to form the clip, the two clipping arms are coplanar, whereas the two parts of the hinge portion are on top of one another and connected together by the spring 250. The spring 250 also serves as a pivot pin for the clipping arms. Each principal component is also provided with a respective engagement fixture (in this case, loop (or cup) type) by which the clip 200.1 may be gripped and opened using a complementary clip deployment tool, e.g. as outlined above.

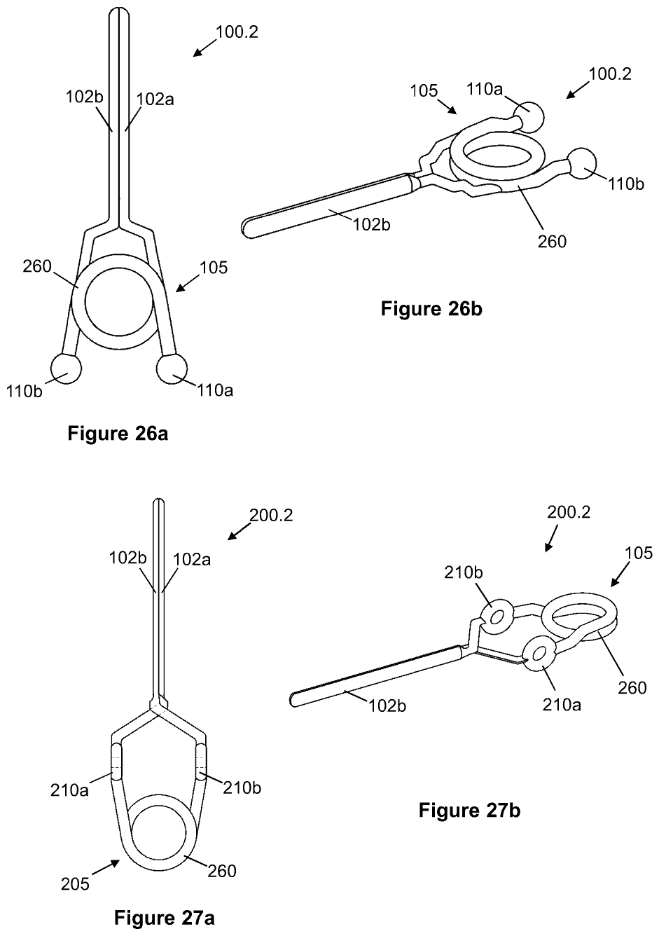

FIGS. 26a and 26b illustrate, in perspective and plan views respectively, an alternative configuration of a clip 100.2, similar to clip 100, but wherein the hinge portion 105 is formed as (or from) a spring coil 260 to which the first and second clipping arms 102a, 102b and the first and second engagement fixtures 110a, 110b are attached. As illustrated, the first and second engagement fixtures 110, 110b are ball-like, but these may readily be substituted with loop- (or cup-) shaped engagement fixtures.

FIGS. 27a and 27b illustrate, in perspective and plan views respectively, a variant 200.2 of the clip of FIGS. 26a and 26b, with the hinge portion 105 again being formed as (or from) a spring coil 260. Again, the clip 200″ has the first and second engagement fixtures 210a, 210b attached to the spring coil 260, but in this case the first and second engagement fixtures 210a, 210b are located between the spring coil and the respective first and second clipping arms 102a, 102b. That is to say, the first and second clipping arms 102a, 102b are respectively attached to the first and second engagement fixtures 210a, 210b; and the first and second engagement fixtures 210a, 210b are attached to the spring coil 260. From the figures it should also be noted that the clip members cross over one another between the between the engagement fixtures 210a, 210b and the clipping arms 102a, 102b. This configuration enables the entire clip to have a compact unitary form. As illustrated, the first and second engagement fixtures 210a, 210b are loop-like, but these may readily be substituted with ball-like engagement fixtures.

FIGS. 28a, 28b and 28c illustrate (in plan view in a closed configuration, in a perspective view in the closed configuration, and in plan view in an open configuration, respectively) a variant 100.3 of the clip 100 of FIGS. 18a-18d. With this clip 100.3, each of the first and second engagement fixtures 110a, 110b has a base portion 110a′, 110b′ that extends around at least part of the first outer member 104a and the second outer member 104b respectively. The main purpose of the base portions 110a′, 110b is to make the attachment point of the engagement fixtures 110a, 110b further around the outer members 104a, 104b, such that applying a lateral force to the bring the engagement fixtures 110a, 110b closer together not only compresses the “V” portion but also flexes (i.e. “pulls” or “spreads”) the outer members 104a, 104b. This enables wider opening of the clip to be achieved.

FIGS. 29a, 29b and 29c illustrate a variant 100.4 of the clip 100.3 of FIGS. 28a-28c, in corresponding views, showing that a subtle thinning of the first and second outer members 104a, 104b of the hinge portion enables wider opening of the clip. More particularly, in FIG. 28c it can be seen that the material comprising the “V” portion is thinner than the material of the outer members 104a, 104b, leading to the “V” doing “most of the work” during the flexing. On the other hand, in FIG. 29c it can be seen that the outer members 104a, 104b are thinner relative to the “V” portion, allowing more of the flexing to happen in the outer members 104a, 104b. Through optimization of the design, a wider opening of the clip is able to be achieved through combined flexing of the “V” and the outer members 104a, 104b, for the same distance of travel of the engagement fixtures 110a, 110b.

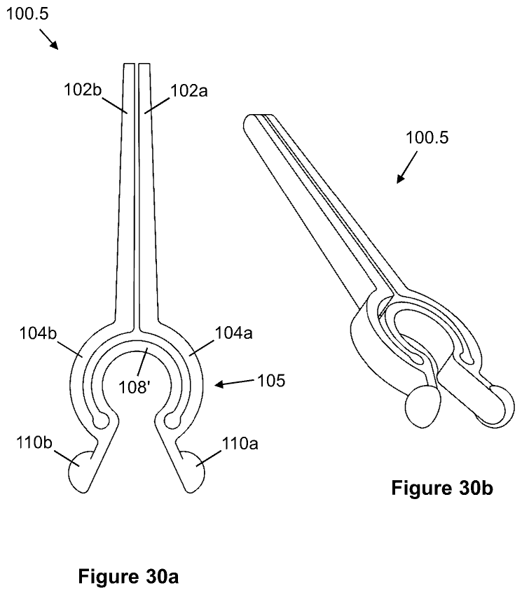

Finally, FIGS. 30a and 30b illustrate (in plan and perspective views, respectively) a further variant 100.5 of the clip 100 of FIGS. 18a-18d. With this clip 100.5, the hinge portion 105 incorporates an arced (rather than V-shaped) resilient hinge member 108′, parallel to the first and second outer members 104a, 104b. This hinge geometry, which represents a development on the part of the inventors over the above V-shaped geometry, provides an open back to the clip 100.5, to reduce the risk of snagging by the deployment tool during use. This also decreases material stress during opening of the clip, by spreading the load of the applied force throughout the arc of the hinge member 108′, rather than concentrating it at the tip of the “V” as the previous V-shaped hinge member does.

In summary, with the various clip configurations described above, the deployment tool tips and the engagement fixtures of the clip act as a single jointed interface employing a ball-and-socket type hold, with a range of socket-like enclosures being possible (e.g. ring, cup, and other geometries). This jointed interface provides a number of mechanical and clinical benefits as compared to existing systems, including but not limited to:
a more robust and solid hold of the clip;
more intuitive and confident manipulation of the clip using the applicator, even in reduced visibility, closed surgical spaces which afford low manoeuvrability; and
near 360-degree engageability about one axis, as well as some misalignment flexibility in all directions, thereby facilitating maximal degrees of freedom for confident clip placement, removal and repositioning.

Moreover, the compliant planar body design of the above-described clips in which the hinge portion and the first and second clipping arms are coplanar and of unitary construction (e.g. as shown in FIGS. 18a-18d, 19a-19d, 28a-28c, 29a-29c, and 30a-30b) is advantageous in that it allows for a lower profile clip design (useful in stacking), higher anatomical visibility, and lack of any friction or surface wear. In the case of the clip of FIGS. 30a-30b in particular, it also allows for an open back of the clip, to avoid tool snagging.

A “pre-fixed” hold enabled by detents such as “dimples-and-pimples” or linear “bumps-and-grooves” around the jointed interface between the tool tips and the engagement fixtures of the clip is also possible, allowing the clip to be set at a desired angle relative to the long axis of the instrument, specific to the angle of approach for the surgical site being treated. Alternatively, or in addition, surface roughening may be provided on the tool tips and/or the engagement fixtures of the clip, to provide more friction and thus a more secure hold.

Surgical Kits

A surgical kit is also provided by the present work, comprising:
a hand-held surgical tool comprising an actuator handle as described above, and
an end effector attached to, or attachable to, the body part of the actuator handle.

A variety of interchangeable end effectors, for different purposes, and/or in different shapes and sizes, are possible within such a kit.

Such a kit may also comprise one or more surgical clips as described above.

Indeed, although the present actuator handles are in no way limited to use with the present clips, when the present handles and clips are used in combination they provide a number of synergistic advantages, including:
optimised ergonomic control at the surgical target, with minimal interaction with surrounding tissue during tool placement (which is especially important when performing neurosurgery);
adjustable angulation for clipping the anatomical feature (e.g. aneurysm neck) in question; and
the ability to deploy the clip in a steady manner with minimal disturbance, and without obstructing the surgeon's view of the surgical site.

### MODIFICATIONS AND ALTERNATIVES

Detailed embodiments and some possible alternatives have been described above. As those skilled in the art will appreciate, a number of modifications and further alternatives can be made to the above embodiments whilst still benefiting from the inventions embodied therein.

For example, although the clipping arms of the presently-described clips are illustrated as being straight, this need not be the case, and alternative embodiments may be realised in which the clipping arms are curved or angular, or have an otherwise complex shape.

Moreover, as those skilled in the art will appreciate, optional features described above in relation to certain embodiments (such as an attachment mechanism for detachably attaching the end effector to the body part of the tool, a rotation mechanism for rotating the end effector relative to the body part of the tool, and/or a locking mechanism for reversibly locking the configuration of the end effector) may naturally be applied to any of the other embodiments, in any viable combination.

Finally, other clips may also be realised, substantially the same as those described herein, but having alternative engagement fixtures (i.e. that are not at least part of a ball, or at least part of a loop). Whilst not benefitting from the above-described advantages conferred by a “ball-and-socket” manner of engagement, such clips would nevertheless benefit from the other advantages described above in relation to the present clips.

1. An actuator handle for a hand-held surgical tool, the actuator handle comprising:
a body part adapted to be gripped by a user's hand, the body part having an end effector mounted or mountable thereon;
an actuating member alongside the body part and arranged to be operably moveable by a single finger of the said hand, in a substantially linear manner between first and second positions relative to the body part, for actuating the end effector in use; and
one or more coupling components coupled to the actuating member, for mechanically transmitting motive force from the actuating member to the end effector upon movement of the actuating member between the first and second positions by the user in use.

2. The actuator handle according to claim 1, wherein the first position is outward relative to the body part and the second position is inward relative to the body part;
optionally wherein the actuating member is resiliently biased into the first position.

3. (canceled)

4. The actuator handle according to claim 1, wherein the actuating member comprises a lever arm;
wherein the coupling components include linkages that, together with the actuating member, form a Scott-Russell linkage mechanism arranged to provide reciprocating motion of a connection point within said linkage mechanism in response to movement of the actuating member between the first and second positions, to which connection point the end effector is coupled in use; and
wherein at least a principal part of the Scott-Russell linkage mechanism forms a grippable part of the body part;
optionally wherein the principal part of the Scott-Russell linkage mechanism comprises guide regions in which to locate the thumb and middle finger of the user's hand in use, the actuating member being arranged to be operated by the first finger of said hand, or wherein the body part and/or the actuating member comprises one or more finger rings or finger tangs in which to locate one or more fingers or the thumb of the user's hand in use;
optionally wherein the actuating member is formed in a compliant unitary manner with the principal part.

5.-9. (canceled)

10. The actuator handle according to claim 4, wherein a first intermediate linkage is arranged to couple a pivot end of the actuating member to the principal part;
optionally wherein the first intermediate linkage is formed in a compliant unitary manner with both the actuating member and the principal part;
optionally wherein the first intermediate linkage comprises flexible waisted regions joining the first intermediate linkage to the actuating member, and joining the first intermediate linkage to the principal part, or wherein the first intermediate linkage is formed of thinner material than both the actuating member and the principal part, to enable preferential flexure of the first intermediate linkage relative to both the actuating member and the principal part.

11.-13. (canceled)

14. The actuator handle according to claim 4, wherein a second intermediate linkage is arranged to couple a point partway along the length of the actuating member to the principal part;
optionally wherein the second intermediate linkage is formed in a compliant unitary manner with either the principal part or the actuating member.

15. (canceled)

16. The actuator handle according to claim 4, wherein substantially the entire actuation mechanism, including the actuating member and the rest of the Scott-Russell linkage mechanism, is formed as a single unitary part.

17. The actuator handle according to claim 4, having a skeletal structure with no outer casing to the Scott-Russell linkage mechanism.

18. The actuator handle according to claim 1, wherein the coupling components include a cable or rod that is arranged to move axially, in a reciprocating manner with operation of the actuating member between the first and second positions, for actuating the end effector.

19. (canceled)

20. The actuator handle according to claim 18, wherein the coupling components include linkages that, together with the actuating member, form a Scott-Russell linkage mechanism arranged to provide reciprocating motion of a connection point within said linkage mechanism in response to movement of the actuating member between the first and second positions, to which connection point the end effector is coupled in use; and
wherein the cable or rod is coupled to said connection point;
optionally wherein an intermediate linkage is arranged to couple a point partway along the length of the actuating member to the principal part, and the intermediate linkage includes an aperture through which the cable or rod from said connection point passes.

21. (canceled)

22. The actuator handle according to claim 1, wherein the actuating member comprises a push-button;
optionally wherein the coupling components include a cable that is arranged to move axially, in a reciprocating manner with operation of the actuating member between the first and second positions, for actuating the end effector, the push-button is coupled to the rack of a rack-and-pinion mechanism, and the pinion of the rack-and-pinion mechanism is coupled to the cable.

23. (canceled)

24. The actuator handle according to claim 1, wherein the actuating member comprises a rotary lever arm;
optionally wherein the coupling components include a cable that is arranged to move axially, in a reciprocating manner with operation of the actuating member between the first and second positions, for actuating the end effector, and the rotary lever arm is coupled to the cable.

25. (canceled)

26. The actuator handle according to claim 18, wherein the cable passes around a pulley mechanism to provide mechanical advantage between the actuating member and the end effector;
optionally wherein the pulley mechanism comprises one or more nested pulleys.

27. (canceled)

28. (canceled)

29. The actuator handle according to claim 1, further comprising:
an attachment mechanism for detachably attaching the end effector to the body part; and/or
a rotation mechanism for rotating the end effector relative to the body part; and/or
a locking mechanism for reversibly locking the configuration of the end effector.

30. (canceled)

31. (canceled)

32. A hand-held surgical tool comprising an actuator handle according to claim 1, and an end effector attached to, or attachable to, the body part of the actuator handle;
optionally wherein the end effector comprises first and second gripping arms configured to engage with, and apply a lateral force to, first and second engagement fixtures of a surgical clip;
optionally wherein the surgical tool is part of a kit comprising one or more such surgical clips, the or each surgical clip comprising:
first and second clipping arms that are mutually openable and closable;
a biasing part arranged to resiliently bias the first and second clipping arms into a closed configuration; and
first and second engagement fixtures respectively coupled to the first and second clipping arms and arranged such that the first and second gripping arms of the end effector of the surgical tool can grip the clip and open the clipping arms by applying a lateral force to the first and second engagement fixtures;
wherein each of the first and second engagement fixtures comprises at least part of a ball or at least part of a loop or cup, with which each of the arms of the end effector of the surgical tool can engage in a substantially ball-and-socket manner.

33. A surgical clip comprising:
first and second clipping arms that are mutually openable and closable;
a biasing part arranged to resiliently bias the first and second clipping arms into a closed configuration; and
first and second engagement fixtures respectively coupled to the first and second clipping arms and arranged such that first and second gripping arms of a complementary surgical clip deployment tool can grip the clip and open the clipping arms by applying a lateral force to the first and second engagement fixtures;
wherein each of the first and second engagement fixtures comprises at least part of a ball or at least part of a loop or cup, with which each of the arms of said surgical clip deployment tool can engage in a substantially ball-and-socket manner;
optionally wherein the first and second engagement fixtures are arranged to be urged towards one another to open the clipping arms, or wherein the first and second engagement fixtures are arranged to be urged apart from one another to open the clipping arms;
optionally wherein the surgical clip further comprises a hinge portion by means of which the clipping arms are openable and closable, optionally wherein the hinge portion comprises the biasing part, or wherein the hinge portion is the biasing part;
optionally wherein the surgical clip is at least partially made of a transparent material, for example along at least part of the first and second clipping arms;
optionally wherein the surgical clip is an aneurysm clip.

34.-38. (canceled)

39. The surgical clip according to claim 33, wherein the hinge portion comprises:
first and second outer members that are respectively attached to the first and second clipping arms, and to which the first and second engagement fixtures are respectively attached; and
a resilient hinge member between, and coupled to, the first and second outer members, and by means of which the first and second outer members, and thence the first and second clipping arms, can move upon application of lateral force to the first and second engagement fixtures;
optionally wherein the resilient hinge member is substantially V-shaped, and wherein the V-shape is closable upon application of sufficient lateral force to the first and second engagement fixtures, resulting in the clip reaching a state of maximal opening;
optionally wherein each of the first and second engagement fixtures has a base portion that extends around at least part of the first outer member and the second outer member respectively;
optionally wherein each of the first and second engagement fixtures incorporates a reinforcing member;
optionally wherein the resilient hinge member is arc shaped;
optionally wherein the first and second outer members are arc shaped;
optionally wherein the first and second outer members are relatively inflexible in comparison to the resilient hinge member, or wherein the first and second outer members are relatively flexible in comparison to the resilient hinge member.

40.-46. (canceled)

47. The surgical clip according to claim 33, wherein the hinge portion and the first and second clipping arms are coplanar; and/or
wherein the hinge portion and the first and second clipping arms are of unitary form.

48. (canceled)

49. The surgical clip according to claim 33, wherein:
the first clipping arm and the first engagement fixture are part of a first principal component;
the second clipping arm and the second engagement fixture are part of a second principal component;
the first and second principal components are moveable relative to one another; and
the biasing part further comprises a spring to which the first and second principal components are both attached.

50. The surgical clip according to claim 33, wherein the hinge portion is formed as a spring coil to which the first and second engagement fixtures and the first and second clipping arms are attached;
optionally wherein the first and second engagement fixtures are located between the hinge portion and the respective first and second clipping arms.

51. (canceled)

52. The surgical clip according to claim 33, wherein:
each of the first and second engagement fixtures comprises at least part of a ball, the shape of which is at least partially spherical, or the shape of which is at least partially a geometric solid having rotational symmetry; or
each of the first and second engagement fixtures comprises at least part of a loop, the shape of which is at least part of a circle, or the shape of which is at least part of a geometric shape having rotational symmetry, such as a triangle, square, hexagon or octagon.

53.-60. (canceled)
